(19) United States
(12) Patent Application Publication
(10) Pub. No.: US 2010/0323444 A1
(43) Pub. Date: Dec. 23, 2010

(54) CULTURING AND DIFFERENTIATING NEURAL PRECURSOR CELLS

(75) Inventors: Dennis A. Steindler, Gainesville, FL (US); Bjorn Scheffler, Remagen-Oberwinter (DE); Antje K. Goetz, Gainesville, FL (US); Noah Walton, Gainesville, FL (US)

Correspondence Address: EDWARDS ANGELL PALMER & DODGE LLP, P.O. BOX 55874, BOSTON, MA, 02205, US

(73) Assignee: UNIVERSITY OF FLORIDA RESEARCH FOUNDATION, Gainesville, FL (US)

(21) Appl. No.: 12/824,872

(22) Filed: Jun. 28, 2010

Related U.S. Application Data
(62) Division of application No. 10/983,112, filed on Nov. 5, 2004, now abandoned.
(60) Provisional application No. 60/518,226, filed on Nov. 7, 2003.

(51) Int. Cl. C12N 5/00

(52) U.S. Cl. 435/377; 435/395

(57) ABSTRACT

Systems and methods have been developed for large-scale propagation and differentiation of populations of neurons and glia from neural precursor cells derived from postnatal brain. Under culture conditions containing pituitary extract and mitogenic factors, cells derived from neural stem cells can be attached to a substrate, maintained and serially passaged in culture. Upon removal of mitogenic factors, clusters of neural progenitor cells can be induced that co-express markers of neural stem cells and immature neurons. Unlimited numbers of cells at characterized stages of neurogenesis can be produced. Upon maturation, neuronal cells extend processes and differentiate into mature neuronal phenotypes capable of generating action potentials.

### CROSS-REFERENCE TO RELATED APPLICATIONS

This application is a Divisional of U.S. application Ser. No. 10/983,112 filed Nov. 5, 2004 that claims priority to U.S. provisional application Ser. No. 60/518,226 entitled “Culturing and Differentiating Neural Precursor Cells,” filed Nov. 7, 2003, the contents of which are incorporated herein in their entireties.

### STATEMENT REGARDING FEDERALLY SPONSORED RESEARCH

The present invention was made with United States government support under grant numbers NIH/NINDS NS37556, HL70143, and T32HDO43730. Accordingly, the United States government has-certain rights in the invention.

### FIELD OF THE INVENTION

The invention relates generally to the fields of developmental biology, neuroscience, stem cells, and regenerative medicine. More particularly, the invention relates to systems and methods for culturing and differentiating neural precursor cells in unlimited quantities, and cellular compositions obtained thereby.

### BACKGROUND

Stem cells have recently been the subject of intense interest because of their potential for treating a wide range of debilitating diseases. A stem cell is a primitive cell that can give rise to a population of progenitor cells. Progenitor cells in turn, depending on their origin, can produce various cell types within one or more lineages. Stem cell populations exist in very small numbers in bone marrow, cord blood, and fetal liver but have also been isolated from skin, muscle and brain tissue. Stem cells are the basis of homeostasis in many tissues and organs by virtue of their self-renewing ability and pluripotency.

Neural stem cells, also termed “neural precursor cells,” that can develop into neurons and glia are of interest because of their potential use in treatments of injury or diseases of the nervous system, particularly Parkinson's disease, Alzheimer's disease and spinal cord injury. The isolation of neural stem cells, however, remains difficult, in part because they make up an exceedingly small fraction of the cell population within tissues. They are difficult to unambiguously identify and are usually identified by testing their capacity for self-renewal and differentiation. Stem cells are usually present in a heterogeneous cell population making identification of an individual stem cell, and determining its characteristics, difficult.

Previous methods for isolating neural precursor cells from postnatal brain have relied on the clonogenicity of these cells. In particular, a clonal neurosphere assay was developed in the mid 1990s that provided a method for clonally expanding a single cell to a mass of cells (termed a “neurosphere”) in suspension culture (see, for example, Kukekov et al., Glia 21:399-407, 1997). Culturing a single cell suspension made from a dissociated neurosphere leads to the generation of secondary neurospheres. Although plated primary and secondary neurospheres can give rise to neurons, astrocytes and oligodendrocytes, these cells are produced in such small numbers as to be impractical for large scale use as therapeutics. Furthermore, because differentiation of individual cell types occurs asynchronously within neurospheres, uncertainty exists as to the time of appearance of particular cell types. It would be desirable to have a culture system that can reproducibly generate large numbers of neural precursor cells, and differentiated neurons and glia derived from such cells.

### SUMMARY OF THE INVENTION

The invention provides methods for in vitro propagation and differentiation of previously unattainable numbers of neural precursor cells derived from neural stem cells of the adult brain. In a unique culture system, neurogenesis can be accurately recapitulated in vitro, from stem cell to functionally mature neuron, and enriched populations of cells at distinct, charcterizable stages in the neurogenic process can be produced in virtually unlimited numbers. The cells can be expanded in tissue culture, induced to proliferate and differentiate in a synchronous fashion, and frozen for later use at any stage during proliferation or differentiation in vitro.

Whereas previous attempts to cultivate neural precursor cells in adhesive culture conditions have been unsuccessful, it has been discovered that growth media containing a combination of pituitary extract and mitogens such as EGF and bFGF permit attachment to a substrate of single cells dissociated from tissues containing neural precursor cells. Following attachment to the substrate, large-scale expansion of neural precursor cells can be induced reproducibly. By manipulating the culture conditions, the cells can be induced to proliferate and differentiate synchronously, generating virtually unlimited numbers of cells at distinct stages in the neurogenic process.

The cultures can provide vast numbers of differentiated neurons and glia, which can be applied as therapeutics for a variety of neurological diseases, as well used for research and diagnostic purposes. For example, it is estimated that propagation of neural precursor cells on solid substrates according to the disclosed methods can generate as many as 40,000-80,000 cells/cm² of surface area, of which about 45-55% are newborn neurons. This high yield of neurons is unprecedented using methods currently known in the art.

As described, large scale cultures containing populations of cells at particular, well-characterized stages in the neurogenic process can be produced and stored at will. The availability of such cultures, greatly enriched in cells at a particular stage of neurogenesis, will be of great benefit in determining the optimal characteristics of neural precursor cells useful for transplantation and other therapeutic uses. Understanding of neurogenesis in the adult central nervous system (CNS) in vivo is still in its infancy. Recently it has been shown that dedicated glial cells give rise to new neurons throughout life (Doetsch, F., Nature Neurosci. 6:1127, 2003). Although neurons and glia are both derived from the embryonic neuroepithelium, sharing common signaling pathways and downstream transcription factors during development (Rowitch, D. H., Nat Rev Neurosci 5:409, 2004), it remains unknown how one major cell class in the adult brain can transpose into another. Establishment of tissue culture systems that now permit propagation and synchronized differentiation of neural precursor cells, and testing of the in vivo behavior of cells with well characterized phenotypes in vitro will pave the way for the development and therapeutic use of stem cells derived from tissues of the adult central nervous system (CNS). For example, cellular compositions enriched in cells at a particular stage of neurogenesis can be used to identify those cells most receptive to being implanted and integrated into the CNS in normal and diseased states. Having been thus identified, particularly useful cellular compositions can be produced in vast amounts.

In other applications, the in vitro and in vivo behaviors of these cells can be exploited in bioassays for drug and other scientific screenings. This product provides a more convenient material than embryonic stem cells and as described above can provide orders of magnitude more cells than existing adult neural stem cell technologies.

The invention will be useful in numerous other applications. For example, because the cultures and systems described mimic in vivo stem cell activities in the rodent subventricular zone (SVZ) (Alvarez-Buylla and Garcia-Verdugo, 2002), they can be utilized as a new in vitro model system for studying SVZ cells. The invention can further be used as a system for studying adult CNS stem cells, for example to identify new stage-specific markers of neuropoeisis, which presently are limited in number and specificity.

Accordingly, and in one aspect, the invention provides a method for culturing neural precursor cells. The method includes the steps of: (a) isolating tissue comprising neural precursor cells from an animal subject; (b) dissociating the tissue to single cells; and (c) attaching the single cells to a substrate in a medium containing pituitary extract and mitogenic factors EGF and bFGF, for a duration of time sufficient to produce a culture containing at least one cell type that is proliferating and/or differentiated.

A variation of the method useful for inducing differentiation of the neural precursor cells into neurons and glia further includes the step (d) of culturing the cells in medium lacking mitogenic factors.

In preferred embodiments of the above methods, the medium contains N2 components.

By appropriate adjustment of the culture conditions and duration of culture in the media, the methods of the invention can be used to produce cell cultures comprising expanded populations of proliferating and/or differentiated cell types (neuronal and glial) at distinct stages of neurogenesis. Within the cultures, the cells respond to the culture conditions and develop synchronously, resulting in cellular compositions having substantial enrichment of cells at a particular stage of neurogenesis at any given time.

Accordingly, and in another aspect, the invention provides cellular compositions comprising neural precursor or neural progenitor cells, or differentiated neurons derived from these cells, in which the composition is enriched in cells at a single stage of neurogenesis. In the practice of the methods of the invention, a wide variety of cell types can be obtained.

In one embodiment of the cellular composition, the enriched cell type is an immature precursor cell (also termed a “trapezoidal cell”) that expresses phenotypic markers of both neural stem cells and glial cells, but not markers of neuronal cell lineage. In some versions of the method used to produce these cells, the immature precursor cell can be expanded (passaged in culture) prior to plating in step (c) above.

In another embodiment, the cell type is a rapidly dividing intermediate cell (also termed a “teardrop cell”) that is produced in culture about 1 day after withdrawal of mitogenic factors, and expresses both phenotypic markers of neural stem cells and phenotypic markers of neuronal lineage, but not the glial cell marker GFAP. Some embodiments of the intermediate cells express the neural stem cell marker nestin, the immature glial cell marker A2B5 and the early neuronal marker β-III tubulin. Other embodiments of these cells express nestin and markers of early neuronal lineage including β-III tubulin and Dlx-2, but not markers of later neuronal lineage including MAP2, NeuN, and GAD.

In versions of the methods involving withdrawal of mitogenic factors, another cellular embodiment that can be obtained is a SVZ progenitor cell (also termed a “phase dark” cell, or neuroblast) that appears in culture about 2-4 days after withdrawal of mitogenic factors. Populations of these cells express both phenotypic markers of neural stem cells and phenotypic markers of neuronal lineage, but do not express the glial cell marker GFAP. The phase dark SVZ progenitor cells can express nestin and markers of early neuronal lineage including β-III tubulin and Dlx-2, and at least one marker of later neuronal lineage such as PSA-NCAM, MAP2, NeuN, or GAD.

In some versions of the methods, the medium in step (d) further comprises retinoic acid, which is added to the culture medium following withdrawal of mitogenic factors. This step results in the appearance of several types of differentiated neurons and glia. Accordingly, in some variations of this method, the differentiated cell type can be a neuron that is a capable of generating an action potential. The cell type can be a bipolar cell. A GABAergic neuron that expresses glutamic acid decarboxylase (GAD) can also be produced by the methods of the invention.

The above-described methods can also be used to generate cellular compositions containing enriched in differentiated glial cells such as astrocytes and oligodendrocytes.

In one preferred embodiment of a cellular composition of the invention, the composition is enriched in proliferating immature precursor cells characterized as: GFAP^{low+}/A2B5⁺/nestin⁺/Dlx-2⁻/β-III tubulin⁻.

In another version, the cellular composition is enriched in rapidly dividing intermediate cells characterized as: GFAP⁻/A2B5⁺/nestin⁺/Dlx-2^{+/-}/β-III tubulin⁺.

In yet a further embodiment, the cellular composition is enriched in SVZ progenitor cells characterized as: GFAP⁻/A2B5⁻/nestin⁺/Dlx-2⁺/β-III tubulin⁺/PSA-NCAM⁺.

Yet another cellular composition is enriched in differentiated neurons.

Other aspects of the invention are discussed below. The invention may be better understood by reference to one or more of the following drawings in combination with the detailed description of specific embodiments presented herein. Unless otherwise defined, all technical terms used herein have the same meaning as commonly understood by one of ordinary skill in the art to which this invention belongs.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

The drawings form part of the present specification and are included to further demonstrate certain aspects of the present invention.

FIG. 1A is a fluorescence micrograph showing immature precursor cells (“trapezoidal” cells) in vitro immunostained using antibodies against A2B5 and GFAP. Cell nuclei are stained with DAPI. Large astrocytes are stained positively for GFAP. Abundant small trapezoid-shaped cells (arrow) are positive for A2B5. Many small cells co-express GFAP and A2B5 (arrowhead). Scale bar—50 μm.

FIG. 1B is a fluorescence micrograph showing a three-dimensional reconstruction of a culture as in FIG. 1A, immunostained with antibodies against nestin and GFAP. Trapezoidal cells express nestin and lie beneath a population of GFAP astrocytes.

FIG. 1C is an electron micrograph of a culture as seen in FIG. 1B showing ultrastructure of trapezoidal cells. Scale bar—10 μm.

FIG. 1D is an electron micrograph showing the boxed area in FIG. 1C at higher magnification. The trapezoidal cells possess glial characteristics, including prominent intermediate filaments (arrowheads). Scale bar—2 μm.

FIG. 1E is two graphs (left, middle) showing whole cell patch clamp electrophysiological recordings of trapezoidal cells (voltage clamp and current clamp, respectively) and a DIC photomicrograph (right) showing trapezoidal cells and a patch clamp electrode.

FIG. 1F is a fluorescence micrograph showing SVZ cells cultivated in differentiating conditions, four days after withdrawal of growth factors. The inset shows the appearance of clusters of “phase dark” SVZ progenitor cells as seen by phase contrast microscopy. Condensed nuclei of these cells are brightly stained by DAPI. Scale bar—20 μm.

FIG. 1G is a fluorescence micrograph of the cells shown in FIG. 1F, which are immunostained with antibodies against PSA-NCAM and Dlx-2. These cells are positive for Dlx-2, and most co-express Dlx-2 and PSA-NCAM. Scale bar—20 μm.

FIG. 1H is an electron micrograph of phase dark cells as shown in FIGS. 1F and 1G. The phase dark cells overlie trapezoidal cells at this stage and are strikingly similar to type A and type C cells of naïve SVZ. Scale bar—5 μm.

FIG. 1I is an electron micrograph showing trapezoidal cells that underlie phase dark cells as shown in FIG. 1H. Scale bar—20 μm.

FIG. 1J is two graphs (left, middle) showing electrophysiological recordings of phase dark cells (voltage and current clamp, respectively) and a DIC photomicrograph (right) showing phase dark cells and a patch clamp electrode. Phase dark cells display membrane properties similar to those of SVZ-born neural progenitors in vivo.

FIG. 2A is three fluorescence micrographs showing phase dark cells induced to terminally differentiate using retinoic acid, immunostained using antibodies against nestin (left), β-III tubulin (middle) and GFAP (right) 4 days after withdrawal of growth factors. Merged image (right) shows co-expression of nestin and β-III tubulin, but not GFAP by these cells. Scale bar—25 μm.

FIG. 2B is three fluorescence micrographs as described in FIG. 2A showing phase dark cells extending bipolar processes 9 days after withdrawal of growth factors as described. Phase dark cells at this stage, corresponding to immature bipolar cells, are negative for GFAP and for nestin, but continue to exhibit β-III tubulin expression. Scale bar—25 μm.

FIG. 2C is six graphs showing voltage clamp characterization of TEA-sensitive K⁺-mediated delayed rectifying currents in bipolar cells 9 days after growth factor withdrawal. In some cells (Cell 1), TEA application exposes underlying K_{A}-mediated currents.

FIG. 2D is a DIC photograph showing appearance of a mature neuron (granular cell) after 28 days in vitro following withdrawal of growth factors. Scale bar—30 μm.

FIG. 2E is a fluorescence micrograph showing mature neurons having a GABAergic phenotype generated in vitro 28 days after withdrawal of growth factors. The cells are immunostained with antibodies against GAD65/67 and β-III tubulin. Cell nuclei are stained with DAPI. Scale bar—30 μm.

FIG. 2F is two graphs showing electrophysiological recordings (left, current- and right, voltage-clamp traces) 28 days after induction of differentiation. Neurons fire a series of TTX-sensitive action potentials.

FIG. 2G is two graphs of electrophysiological recordings as described in FIG. 2F. Traces on the left show that spontaneous synaptic activity can be recorded, and entirely blocked by application of picrotoxin (PIC; right traces).

FIG. 3A is four micrographs from a series showing real-time microscopy of SVZ cells (passage 3). The arrow highlights an area where a series of rapid mitotic events leads to a large cluster of phase dark cells in a period of only 27 h. The sequence starts at 24 h after growth factor withdrawal. Scale bar—40 μm.

FIG. 3B is three micrographs showing phase contrast (left), fluorescence (middle) and combined phase contrast with overexposed fluorescence (right) images of phase dark cells 48-72 hours following induction of differentiation. Fluorescence shows more than 90% labeling of phase dark cells two days after exposure to BrDU. Scale bar—25 μm.

FIG. 3C is two phase contrast micrographs (upper and lower left) showing clonal neurospheres (NS) derived from cultured P8 and adult SVZ cells, respectively, and two graphs (upper and lower right) showing number of NS formed (upper) and relative change of NS frequency (lower) following withdrawal of growth factors. Scale bar—200 μm.

FIG. 3D is a fluorescence micrograph showing neurons derived from neurospheres, immunostained with antibodies against β-III tubulin. Scale bar—20 μm.

FIG. 3E is a fluorescence micrograph showing glia derived from neurospheres, immunostained with antibodies against CNPase. Scale bar—20 μm.

FIG. 4A is a phase contrast micrograph (left) and two graphs of electrophysiological recordings (middle and right, voltage clamp and current clamp, respectively) showing distinctive morphological and electrophysiological characteristics of rapidly dividing intermediate cells that appear in culture 24 h after induction of differentiation. Scale bar—15 μm.

FIG. 4B is a fluorescence micrograph showing intermediate cells as described in FIG. 4A, immunostained with antibodies against the immature glial marker A2B5. Arrow indicates a dividing cell that expresses A2B5. Scale bar—15 μm.

FIG. 4C is a fluorescence micrograph showing clusters of β-III tubulin positive intermediate cells (“teardrop” cells) with characteristic teardrop morphology. Scale bar—20 μm.

FIG. 4D is a fluorescence micrograph of teardrop cells stained with DAPI, which reveals a condensed nucleus and bright DAPI label.

FIG. 4E is a fluorescence micrograph showing teardrop cells immunostained with antibodies against A2B5 and the neuronal marker β-III tubulin. Co-expression of these two markers is the hallmark of this transient phenotype, and levels of co-expression differ between individual clusters and cells. The inset highlights a relationship between β-III tubulin filaments and the spindle apparatus in the dividing cell shown. Scale bar—5 μm.

FIG. 4F is an electron micrograph showing teardrop cells. These cells have darkened, elongated nuclei, and cell and nuclear sizes that are intermediate between proliferating glia and phase dark cells. Scale bar—1 μm.

FIG. 4G is an electron micrograph of a teardrop cell showing cytosolic vacuoles and large numbers of mitochondria and free ribosomes, but lacking intermediate filaments. Scale bar—0.5 μm.

FIG. 4H is an electron micrograph taken at higher power than FIG. 4G showing details of teardrop cell morphology including absence of intermediate filaments.

### DETAILED DESCRIPTION OF THE INVENTION

The invention relates to the development of a system and method for propagating virtually unlimited numbers of self-renewing neural precursor cells from postnatal brain that can be induced to differentiate into functional neurons and glia, and cellular compositions highly enriched in these cells. As discussed, it has been discovered that growth media containing a combination of pituitary extract and mitogens such as EGF and bFGF permit attachment to a substrate of single cells dissociated from tissues such as the subventicular zone (SVZ) of the brain that contain neural precursor cells. Following attachment to the substrate, large-scale expansion of the neural precursor cells can be achieved reproducibly. By appropriately manipulating the culture conditions, the cells can be induced to proliferate and differentiate synchronously, generating vast numbers of cells at distinct stages in the neurogenic process.

The cells in the cultures are capable of differentiating into mature neurons and glia. Using the novel culture conditions defined herein, a multipotent cell derived from the postnatal brain, resembling an immature glial phenotype, can be propagated in unlimited numbers and induced at will in vitro to give rise to neurons and glia. This cell culture system provides a new and highly efficient method to expand the rare self-renewing neural precursor cells from the postnatal CNS, and the first method to generate cellular compositions highly enriched in well characterized cells at distinct stages of the neurogenic process.

In one aspect, the invention provides a method or system for culturing neural precursor cells in vitro. The method includes the steps of: (a) isolating tissue comprising neural progenitor cells from an animal subject; (b) dissociating the tissue to single cells; and (c) attaching the single cells to a substrate in medium comprising pituitary extract and mitogenic factors EGF and bFGF, for a duration of time sufficient to produce a culture comprising at least one cell type that is proliferating and/or differentiated.

Methods of isolating tissue comprising neural progenitor cells, for example from the SVZ of an adult animal, are known and, as described in Examples below, briefly involve removing the brains from the animals and placing them in culture medium (for example N5 medium described below) for dissection. Typically the lateral ventricles are exposed by coronal sectioning, and the surrounding tissue is microdissected from the brain slices. Following mincing of the tissue into pieces of about 1 mm³, the tissue pieces are triturated, for example using fire-polished glass pipettes of decreasing widths, in trypsin solution (for example, 0.005%-0.25%, pH 7.3, at 37° C. for 15-25 min) to obtain a cell dissociate containing primarily single cells.

For the practice of the invention, following trituration, the single cell dissociate is centrifuged and the cell pellet is then plated in uncoated plastic tissue culture dishes (for example multiwell cell culture dishes or T-75 flasks) in growth medium at a density of at least 50,000 cells/cm², and then cultured overnight in a humidified incubator at 37°. This step allows for attachment of the cells to the substrate.

Without intending to be bound to any particular theory, it is believed that the inclusion of EGF and bFGF, and in particular pituitary extract (for example, bovine pituitary extract), in the growth medium is required for, or greatly promotes, the attachment of neural precursor cells to solid substrates such as plastic surfaces commonly used for adhesive culture systems. Attachment of these cells to a substrate permits the large scale expansion of these cells. Heretofore, it has been very difficult to achieve attachment of neural precursor cells in culture. Accordingly, such cells were previously grown in suspension cultures, wherein floating single cells proliferated to form spheres of cells (“neurospheres”) containing heterogeneous phenotypes representing progeny of the original progenitor cell at various stages of differentiation.

As described above, the use of a substrate for cell attachment, as opposed to a suspension culture in which cells proliferate as neurospheres, provides the advantage of greatly expanding the numbers of proliferating cells with potential to differentiate into mature neuronal phenotypes. For example, as discussed, it is estimated that propagation of neural precursor cells on solid substrates can generate up to 40,000-80,000 cells/cm² of surface area, of which about 45-55% are newborn neurons. This number greatly exceeds the yield of neurons previously obtainable from neurosphere cultures.

Preferred basal growth media for the methods and systems of the invention are those media generally known to support the maintenance and proliferation of neural stem cells. Cell culture techniques are generally known in the art and are described in detail in methodology treatises such as Culture of Animal Cells: A Manual of Basic Technique, 4th edition, by R. Ian Freshney, Wiley-Liss, Hoboken, N.J., 2000; and General Techniques of Cell Culture, by Maureen A. Harrison and Ian F. Rae, Cambridge University Press, Cambridge, UK, 1994. The compositions of basal growth media suitable for cell culture of neural precursor cells (for example, DMEM and Ham's F-12) are known to those of skill in the art of cell culture.

Preferred components of growth media suitable for culture and propagation of neural precursor cells include, for example, supplements known as “N2-components,” or “N2 supplements,” which comprise: apo-transferrin, insulin, progesterone, sodium selenite, and putrescine. N2 components can be purchased separately from commercial suppliers of tissue culture media and supplements, or tissue culture supplements containing pre-mixed N2-components at selected concentrations are commercially available, for example from suppliers such as Hyclone and R&D Systems.

A particularly preferred growth medium for the purpose of promoting attachment and proliferation of neural precursor cells is “N5” medium, which comprises the following components, used at the concentrations indicated:

DMEM/F-12 media (“DF;” Invitrogen, Cat. No. 11320-033)

2% antibiotic-antimycotic (Invitrogen, Cat. No. 15240-062)

5% Fetal Calf Serum (“FCS,” Hyclone, Cat No. SH30031.03)

N2-components:
100 μg/ml human apo-transferrin
5 μg/ml human insulin
20 nM progesterone
30 nM sodium selenite
100 μM putrescine

bovine pituitary extract, 37 μg/ml

basic fibroblast growth factor (bFGF), 40 ng/ml

epidermal growth factor (EGF), 40 ng/ml.

The cells can be cultivated in N5 medium supplemented with FCS as shown, but it has been found that serum is not essential for the practice of the invention.

Following overnight plating, non-adherent cells from the plated suspension of single cells derived from SVZ tissue are collected on the next day from the tissue culture dishes or flasks, and a single cell suspension is prepared as described above from the non-adherent cells. The cells, termed “neural precursor cells,” are then plated at a density of at least 50,000 viable cells/cm² in N5 media and allowed to reach confluency, which generally occurs within 1-4 days of growth under these conditions, with replacement of the medium every other day until confluency. Following proliferation under culture conditions as described above, the neural precursor cells are referred to herein as “neural progenitor cells,” or “proliferating neural progenitor cells.”

A “proliferating cell type,” as used herein, refers to a cell type capable of undergoing DNA synthesis and mitosis. Proliferating cell types can be detected by methods well known in the art, including uptake of radiolabeled thymidine, or incorporation of 5-bromo-2-deoxyuridine (BrdU). Examples of proliferating cell types that can be derived using the culture methods of the invention include but are not limited to various cell types described herein as “neural precursor cells,” “immature precursor cells” (“trapezoidal cells”), “rapidly dividing intermediate cells” (“teardrop cells”), and “SVZ progenitor cells” (or “phase dark cells”).

Conveniently, proliferating neural progenitor cells grown under these conditions can be passaged repeatedly. In general, the cells are passaged by removing the adherent cells with trypsin, and then seeding the cells at densities between 75,000 and 85,000 viable cells/cm² in new culture dishes/flasks. After passaging and at medium changes, mitogens, for example, EGF and FGF, are supplied at concentrations of 40 ng/ml, and can be supplied at reduced concentrations (for example, 20 ng/ml) at two day intervals thereafter.

Most advantageously, at any given stage during the proliferation phase, the cells can be frozen and stored in liquid nitrogen for extended periods of time. The cells can then be thawed and can undergo the same treatment schedule, yielding the same expansion rates as non-frozen specimens without losing their stem cell attributes. For example, it was found that cells frozen at passage 1 were later expandable through serial passaging to passage 20. These cells exhibited identical behavior to cells derived from wild type C57/B6 mice serial passaged similarly without freezing.

At any given time, the proliferating cells of the invention can be induced to differentiate by withdrawal from the culture medium of mitogenic factors (EGF and bFGF) and serum, if used. Under these conditions, clusters of small phase-dark cells reproducibly appear 2-3 days following withdrawal of growth factors from the proliferating cultures. As further described below, such cells initially express markers of immature neurons and later express markers of more differentiated neurons.

As used herein, the term “differentiated neural cell type” is defined broadly and includes any cellular phenotype having neural characteristics that develops in culture from a neural stem, precursor, or progenitor cell cultured under conditions defined and described herein to induce such differentiation. Those of skill in the art will recognize that the transition from a stem-like glial cell (such as an astrocyte from the adult SVZ) to a neuron is a continuum; nevertheless, as shown below, cells expressing subsets of markers indicative of differentiation along a particular lineage can be recognized, isolated and propagated at select stages in the process.

In preferred embodiments of the invention, induction to the differentiated state is achieved by attaching the cells to a substrate, for example a glass coverslip or other suitable tissue culture surface. Preferably the culture surface is coated, for example with poly-L-ornithine (15 μg/ml)/laminin (1 μg/ml), or other suitable mixture. Such surfaces have shown to provide an ideal substrate for cell attachment and neural differentiation and maturation. Use of untreated substrates (for example, glass or plastic alone) results in lack of cellular attachment and/or significantly decreased quantities of inducible neuroblasts.

Functional neurons can be generated in these cultures by a protocol adapted from Song et al. (Nature 417:39, 2002). Briefly, retinoic acid (Sigma-Aldrich, St. Louis, Mo., 0.5 μM) is added between 7 and 10 days after induction of differentiation, and replenished bidaily, for example for a period of 6 days. Cytosine β-_{D}-Arabinofuranoside (Sigma-Aldrich, 0.5 μM) is added for two days following retinoic acid treatment. Neurons are then allowed to differentiate in DF medium supplemented with N2-components, 0.5% FCS and brain-derived neurotrophic factor (BDNF, 20 ng/ml, R&D Systems Minneapolis, Minn.). Media is changed bidaily.

The cells produced by the culture systems and methods of the invention can be characterized by detecting the presence or absence of one or more cell-type specific or cell lineage markers, for example antigens that are expressed in a given cell type or are associated with a particular cell lineage, or in a cell type at a recognized stage in a process of differentiation. A preferred method of detecting an antigenic marker is by immunohistochemistry or immunocytochemistry, whereby an antibody that specifically recognizes (binds to) the marker protein or a portion thereof in the cell is visualized, for example by fluorescence microscopy. As is well known in the art and demonstrated in Examples below, three or more different markers can be detected simultaneously in the same cells by multiply reacting the cells with antibodies directed against the different markers, followed by detecting binding of each of the markers using secondary antibodies labeled, for example, with probes (such as fluorescein, rhodamine, Alexa-555, AMCA, Cy3, Oregon green, and the like) that fluoresce at different wavelengths and hence appear as different colors (typically green, red and blue) when viewed in a fluorescence microscope with appropriate filter sets.

Any appropriate marker of cell type or cell lineage that can distinguish a cell at one stage of differentiation, or of one lineage, from another at a different stage, or of a different lineage, can be used to demonstrate the status of cell differentiation, ranging from multipotent neural precursor cell to fully differentiated neuron or glial cell. Several such markers are recognized as useful in delineating and distinguishing cells of the CNS at different stages of differentiation.

For recognizing cells with stem-like characteristics, a useful marker is nestin. Nestin is a type of intermediate filament known to be expressed in neural precursor cells. A marker that can be used to identify immature glial cells is A2B5, which recognizes an immature neural ganglioside.

A preferred marker of glial cells (which include astrocytes, oligodendrocytes and microglia) generally at any stage of differentiation is glial fibrillary acid protein (GFAP). Markers of a particular class of differentiated glial cells, i.e., oligodendrocytes, include CNPase and 04.

Cells committed to becoming neurons (for example, neuroblasts and more differentiated neuronal classes such as bipolar cells) can be identified by the presence of specific neuronal markers, which are known to include β-III tubulin, MAP2, NeuN, Dlx-2 and PSA-NCAM. The terms “early neuronal markers,” or “markers of early neuronal lineage,” as used herein, refer to phenotypic markers known to be expressed by immature cells committed to differentiate along the neuronal pathway. As an example, β-III tubulin, PSA-NCAM and Dlx-2 are defined as early neuronal markers. “Markers of late neuronal lineage,” as used herein, refer to phenotypic markers expressed in differentiated neurons, for example GABAergic neurons, i.e., neurons expressing the inhibitory neurotransmitter gamma-amino butyric acid (GABA). GABAergic neurons can be identified for example by markers such as the 65 and 67 kDa forms of glutamic acid decarboxylase (GAD65/67), an enzyme that converts glutamic acid to GABA. Other markers of late neuronal lineage include MAP-2 and NeuN.

The cells of the invention can also be characterized by their morphological appearance (such as shape, degree of darkness, etc.) when viewed by microscopy of various types, including but not limited to light microscopic methods such as phase contrast/differential interference (DIC), fluorescence microscopy, inverted phase microscopy, and confocal microscopy, as well as scanning and transmission electron microscopy.

The cells of the invention can also be characterized by their functional attributes, for example as shown below by their electrophysiological recording patterns when subjected to single cell recording techniques such as whole cell patch clamping.

In studies more fully described in Examples below, the phenotypic characteristics of proliferating neural progenitor cells derived from neural precursor cells of the SVZ and of differentiating cells that appear during the process of differentiation that occurs in the cultures following withdrawal of mitogenic factors from the culture media, were extensively characterized. The analysis led to the recognition of several distinct cellular phenotypes with characteristic morphological, immunophenotypic and electrophysiological profiles that could be produced at given times within the cultures. At any particular time, the cultures are highly enriched with cells of a particular phenotype. The characteristics of several main cell types that can be propagated using the methods of the invention are summarized in Table 1 below.

TABLE 1

| | Cell type | | | |
|---|---|---|---|---|
| | Immature precursor cell | | | |
| | (stem cell with glial | Rapidly dividing | | |
| | characteristics; | intermediate cell | SVZ progenitor cell | |
| | “trapezoidal” cell) | (“teardrop” cell) | (“phase dark” cell) | Mature neuron |
| Days after | N/A | 1 | 2-4 | 7-28 |
| GF WD | | | | |
| Immuno- | GFAP^{low+} | GFAP⁻ | GFAP⁻ | GFAP⁻ |
| phenotype | | | | |
| | A2B5⁺ | A2B5^{+/-} | A2B5⁻ | A2B5⁻ |
| | Nestin⁺ | Nestin⁺ | Nestin⁺ | Nestin⁻ |
| | β-III tubulin^{-/low} | β-III tubulin⁺ | β-III tubulin⁺ | β-III tubulin⁺ |
| | PSA-NCAM⁻ | PSA-NCAM⁻ | PSA-NCAM⁺ | PSA-NCAM^{+/-} |
| | Dlx-2⁻ | Dlx-2^{+/-} | Dlx-2⁺ | Not determined |
| | MAP-2a-c⁻ | MAP-2a-c⁻ | MAP-2a-c⁻ | MAP-2⁺ |
| | NeuN⁻ | NeuN⁻ | NeuN⁻ | NeuN⁺ |
| | GAD⁻ | Not determined | Not determined | GAD⁺ |
| Electro- | A-type K⁺ channels. | Intermediate between | More polarized than | Action potentials |
| physiology | No action potential. | proliferating cells and | proliferating cells; | |
| | | phase dark cells. | resemble neuroblasts | |
| | | | in vivo. | |

Accordingly, and in another aspect, the invention provides cellular compositions comprising neural precursor or neural progenitor cells, or differentiated neurons derived from these cells, wherein the composition is enriched in cells at a single stage of neurogenesis. By the term “enriched in cells at single stage of neurogenesis” is meant having a greater proportion of cells at a single stage of neurogenesis than would be present, for example, in a tissue culture system such as a culture containing neurospheres, which are characterized by the presence of heterogeneous populations of neural precursor cells at widely divergent stages of neurogenesis, ranging from stem cell to committed neuron. As described, the disclosed methods for the first time permit the synchronous induction of cells at any given stage of neurogenesis. Accordingly, the majority of the cells in the cultures will develop concurrently, resulting in large populations of cells at the same stage of neurogenesis. Particularly preferred cellular compositions are enriched in cells having distinctive phenotypic profiles. As non-limiting examples, the invention provides cellular compositions enriched in proliferating immature precursor cells characterized as: GFAP^{low+}/A2B5⁺/nestin⁺/Dlx-2⁻/β-III tubulin⁻. Other cellular compositions are enriched in rapidly dividing intermediate cells characterized as: GFAP⁻/A2B5⁺/nestin⁺/Dlx-2^{+/-}/β-III tubulin⁺. Another embodiment of a cellular composition is enriched in SVZ progenitor cells characterized as: GFAP⁻/A2B5⁻/nestin⁺/Dlx-2⁺/β-III tubulin⁺/PSA-NCAM⁺. In yet another variation, the invention provides compositions enriched in differentiated neurons.

The below described preferred embodiments illustrate adaptations of these systems, methods and compositions. Nonetheless, from the description of these embodiments, other aspects of the invention can be made and/or practiced based on the description provided below.

### EXAMPLES

### Example 1

### Material and Methods

The following materials and methods were used in Examples 2-7 described below.

Isolation of SVZ cells. C57/B6 mice (Jackson Laboratories, Bar Harbor, Minn.) and age- and sex-matched transgenic mice expressing nestin-GFP (J. L. Mignone, V. Kukekov, A. S. Chiang, D. Steindler, G. Enikolopov, J Comp Neurol 469, 311, 2004) were housed under standard conditions. To obtain SVZ cells, 8 day-old (P8) and adult (>90 days-old) animals were decapitated and their brains removed and placed in DMEM/F-12 (DF, Invitrogen, Carlsbad, Calif., Cat No. 11320-033) containing 20 mg/ml penicillin, 20 mg/ml streptomycin, and 25 ng/ml amphotericin B (collectively, “antibiotics”). The lateral ventricles were exposed via coronal sectioning, and the surrounding tissue was microdissected from brain slices. Under aseptic conditions, extracted tissues of five animals were pooled, placed in Dulbecco's phosphate-buffered saline (PBS), and manually dissociated into 1 mm³ pieces.

Tissue culture. Tissue pieces prepared as described were triturated in 0.005% trypsin (15 min, 37° C., pH 7.3) and placed overnight in uncoated T75 plastic tissue culture dishes in N5 media: DF containing N2 supplements (modified N-components [N-components include 100 μg/ml human apo-transferrin, 5 μg/ml human insulin, 20 nM progesterone, 30 nM sodium selenite, 100 μM putrescine], supplemented with 37 μg/ml pituitary extract, 40 ng/ml EGF and FGF, 1% antibiotics, and 5% fetal calf serum (FCS, HyClone, Logan, Utah). Unattached cells were collected, gently triturated using fire-polished pipettes and replated onto uncoated plastic dishes. The cells were then allowed to proliferate to confluency in N5 media. EGF and FGF (20 ng) were added bidaily. Confluent cell layers were frozen in aliquots of 1×10⁶ cells and maintained in liquid nitrogen.

For experimentation, cells were thawed and passaged two or more times (a minimum of 5 population doublings) using 0.005% trypsin and N5 media, with bidaily 20 ng/ml EGF/FGF supplementation. To induce differentiation, cells were plated on glass coverslips coated with polyornithine (Sigma, St. Louis, Mo., 10 μg/ml) and laminin (1 μg/ml) (LPO) at densities of approximately 2×10⁵ cells/cm². Cells were proliferated to 90-100% confluency and were induced to differentiate by removing growth factors and serum from culture media. Dividing cells were labeled with BrDU (Sigma, St. Louis, Mo., 10 μg/ml).

Functional neurons were generated by a protocol adapted from Song et al., Nature 417:39, 2002). Briefly, retinoic acid (Sigma-Aldrich, 0.5 μM) was added between 7-10 days after induction of differentiation and replaced bidaily for a period of 6 days. Cytosine β-_{D}-Arabinofuranoside (Sigma-Aldrich, 0.5 μM) was added for two days following retinoic acid treatment. Neurons were then allowed to differentiate in N2 supplemented DF with 0.5% FCS and brain-derived neurotrophic factor (BDNF, 20 ng/ml). Unless otherwise specified, tissue culture plastic ware was obtained from Corning/Costar (Corning, N.Y.), media was from Invitrogen (Carlsbad, Calif.), and growth factors were obtained from R&D Systems (Minneapolis, Minn.). Media was changed bidaily.

Neurosphere assay. Passage 3 cells from P8 and adult SVZ were trypsinized, counted, and resuspended in non-adhesive 6-well plates (Costar) in 2 ml/well of N5 media containing 1% methylcellulose as described (Kukekov V G et al., Exp. Neurol. 156:533, 1999). EGF and FGF (20 ng/ml) were added bidaily. To verify clonality of neurospheres generated under these conditions, serial dilution of SVZ cells was performed from 0.6-20×10³ cells/cm². A linear seeding:neurosphere relationship was observed between densities of 2.5-20×10³ cells/cm². All neurosphere experiments were performed at a seeding density of 10⁴ cells/cm².

To quantify presence of neurosphere-forming cells, total numbers of primary neurospheres were counted per well using bright field microscopy (n=3 for each, adult and P8 cultures, derived from two independent experiments). Secondary neurospheres were derived from dissociated primary neurospheres and evaluated similarly (n=2 for each, adult and P8 cultures, from two independent experiments). Neurospheres were counted 14-21 days after cell seeding. The level of statistical significance was set at p<0.01, and was calculated using the student's t-test. Neurospheres were plated on LPO-coated glass coverslips overnight in N5 media. Cells migrating out of neurospheres were allowed to differentiate for 7 days after removing growth factors and serum from culture media. Individual primary and secondary neurospheres were assessed for multipotentiality by double immunofluorescence analysis with neuronal (i.e., β-III tubulin, MAP 2, NeuN) and glial (i.e., CNPase, GFAP, 04) marker proteins.

Live cell microscopy. Passage 3 SVZ cells were grown to confluency in N5 media on LPO-coated 3 cm glass coverslip dishes (Willco Wells BV, Amsterdam, The Netherlands). Cells were induced to differentiate as described, and were monitored under standard culture conditions (37° C., 5% humidified C0₂) on a Zeiss Cell Observer system (Carl Zeiss Microimaging Inc., Thornwood, N.Y.). Five randomized visual fields (200×) were selected for analysis 24 hours following induction of differentiation. Phase contrast images were taken every five minutes for up to 30 hours. Images were compiled into movies using Axiovision™ software (Zeiss, Gottingen, Germany).

Immunocytochemistry. Cells were fixed for 10 min with 4% paraformaldehyde. After washing with PBS, nonspecific antibody activity was blocked for 20 min in PBS containing 0.01% Triton X-100 (PBS-T), 10% FCS, and 5% goat serum. Primary antibodies were applied 1-2 hr at room temperature, or overnight at 4° C. in PBS-T and 10% FCS.

Primary antibodies included the following: A2B5 (1:150, mouse monoclonal IgM, Chemicon, Temecula, Calif.); β-III tubulin (1:300, mouse monoclonal, Promega, Madison, Wis.; BrDU (1:50, mouse monoclonal, Becton-Dickinson, San Jose, Calif.); CNPase (1:250, mouse monoclonal, Chemicon, Temecula, Calif.), NeuN (1:50, mouse monoclonal, Chemicon); 04 (1:150, mouse monoclonal IgM, Chemicon, Temecula, Calif.); PSA-NCAM (1:400, mouse monoclonal IgM, Chemicon, Temecula, Calif.); Map-2 (1:30,000, chicken polyclonal, gift from Dr. Gerry Shaw); Dlx-2 (1:50, goat polyclonal, Santa Cruz Biotechnology, Santa Cruz, Calif.); GAD 65/67 (1:125, rabbit polyclonal, Santa Cruz Biotechnology); and GFAP (1:600; rabbit monoclonal, DAKO, Carpinteria, Calif.).

Secondary antibodies were applied for 45 min at room temperature in PBS-T and 10% FCS. Secondary antibodies included: Alexa-555 goat anti-chicken (1:300, Molecular Probes, Eugene, Oreg.); AMCA goat anti-rabbit IgG (1:50, Jackson Labs, West Grove, Pa.); Cy3 goat anti-mouse IgG (1:300, Jackson Labs, West Grove, Pa.); Cy3 goat anti anti-mouse IgM (1:600, Jackson Labs, West Grove, Pa.), Oregon Green donkey anti-goat (1:200, Molecular Probes, Eugene, Oreg.), and Oregon Green goat anti-rabbit (1:200, Molecular Probes, Eugene, Oreg.). Nuclei were visualized using a DAPI stain (1 μg/ml, Sigma-Aldrich, St. Louis, Mo.). BrDU detection required a pretreatment of 2 hr in SSC-formamide, 3×10 min washes in SSC, 30 min in 2N HCl, and 10 min in 0.1M borate buffer.

Fluorescence microscopy was performed on a Leica DMLB upright microscope (Leica, Bannockburn, Ill.) and images were captured with a Spot RT Color CCD camera (Diagnostic Instruments, Sterling Heights, Mich.). Three dimensional imaging on some specimens was performed using a fully automated Axiovert 200 inverted microscope equipped with Apotome™ technology and images were reconstructed using Axiovision™ software (Zeiss, Gottingen, Germany).

Electrophysiology. Culture media was removed and cells attached to glass coverslips were placed into a holding chamber continuously perfused with oxygenated artificial cerebrospinal fluid (aCSF) containing: 125 mM NaCl, 3 mM KCl, 26 mM NaHCO₃, 1.25 mM NaH₂PO₄, 20 mM glucose, 1 mM MgCl₂, and 2 mM CaCl₂ maintained at 35° C. during experiments. Cell cultures were visualized using video-enhanced DIC and fluorescence microscopy with a Nikon Eclipse E600FN upright microscope (Nikon, USA).

Patch electrodes were pulled from thick-walled borosilicate capillary glass (WPI, Sarasota, Fla.) to a resistance of 4-6 MO using a Flaming-Brown P-87 microelectrode puller (Sutter Instruments, Novato, Calif.). Intracellular pipette solution was comprised of: 145 mM K-gluconate, 10 mM HEPES, 10 mM EGTA, and 5 mM MgATP (pH 7.2, osmolarity 290).

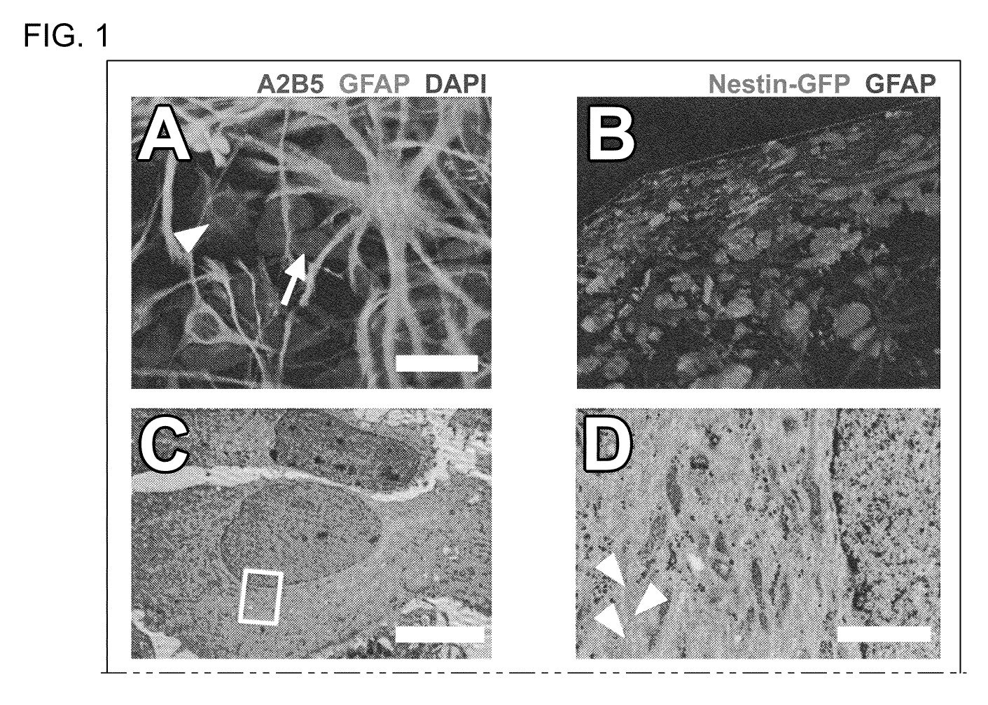

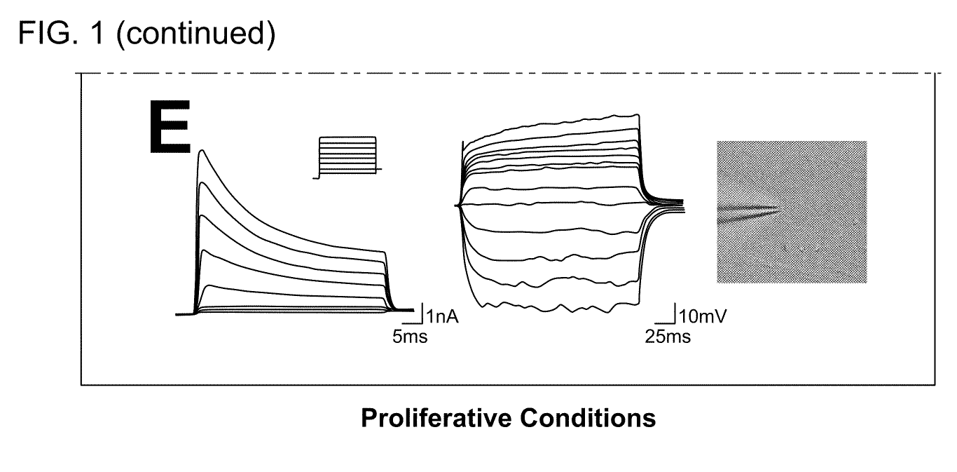

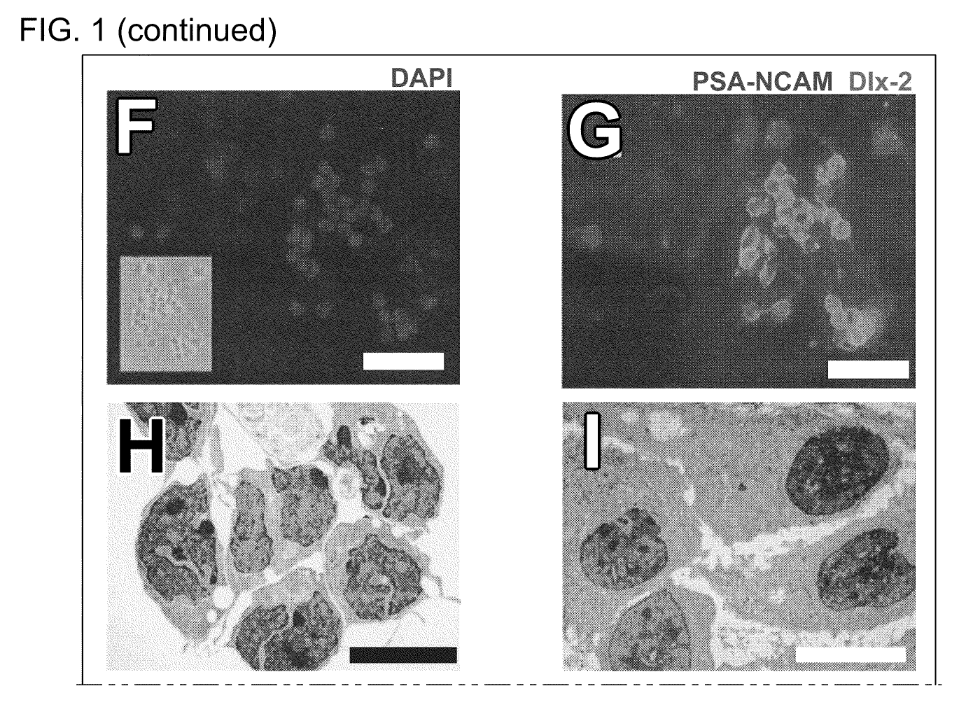

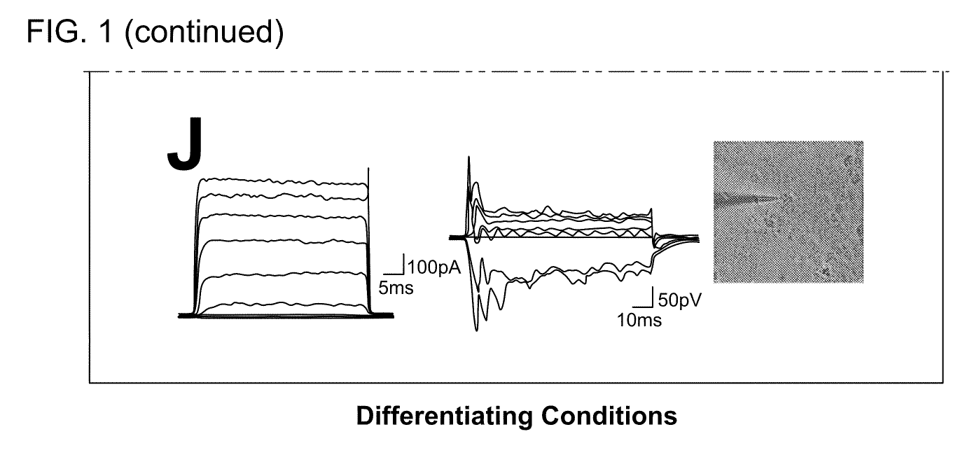

For experiments in which post-synaptic currents were recorded, 145 mM K-gluconate was replaced with 125 mM KCl and 20 mM K-gluconate. Recordings were performed with an Axopatch-1 D™ (Axon Instruments, Union City, Calif.), and filtered at 5 kHz. Clampex™ 8.2 (Axon Instruments, Union City, Calif.) was used to deliver command potentials and for data collection. Series resistances were <20 MO and checked frequently to ensure that they did not deviate. For voltage-clamp experiments, a step protocol was applied which held the membrane at potentials between −80 mV and +60 mV for 50 ms after a pre-pulse period of 200 ms at −100 mV (as shown in FIG. 1E). During current-clamp experiments, a step protocol was utilized in which currents between 10-100 pA were applied per step. Cells in which series resistance was >20 MΩ, or in which series resistance fluctuated, were excluded from studies. Clampfit 8.2 (Axon Instruments, Union City, Calif.) was used to analyze voltage and current traces.

Picrotoxin was applied at a concentration of 50 μM, and Tetrodotoxin was used at 400 nM (Alomone Labs, Jerusalem, Israel). All other chemicals and reagents were obtained from Sigma-Aldrich (St. Louis, Mo.) unless otherwise noted. Data were expressed as mean±standard error of the mean.

Electron microscopy. Passage 3 SVZ cells were grown as described in N5 media on LPO-coated aclar coverslips. Cells were fixed and evaluated prior to differentiation, 24 hours after differentiation, and upon appearance of phase dark cell colonies. Fixation was performed for 30 min at room temperature in PBS containing 2.5% paraformaldehyde, 0.1% sodium cacodylate, and 0.02% glutaraldehyde, followed by treatment with 2% osmic acid in 0.1 M sodium cacodylate at 4° C. for 1 hr. Cells were dehydrated with sequential 10 minute immersions in 30-100% ethanol gradients. Cells were then imbedded in “Spon” epoxy containing LX1 12 (52% v/v), NMA (31% v/v), DDSA (17% v/v), and DMP-30 (catalyst). All reagents for electron microscopy were obtained from Sigma-Aldrich (St. Louis, Mo.).

Epon-embedded specimens were thin-sectioned on a Leica Ultracut™ T ultramicrotome and were counterstained with uranyl acetate and lead citrate. Samples were visualized on a Leica EM1 OA™ transmission electron microscope at magnifications ranging between 1-16,000×. Images were captured using a CCD digital camera (Finger Lakes Instrumentation, Lima, N.Y.).

### Example 2

### Stage I of In Vitro Neurogenesis: Characterization of Cells Under Proliferative Conditions

This example describes characteristics of cells that appear in SVZ cultures maintained under proliferative conditions as described in the Methods above.

Cells were harvested as described from a neurogenic brain region known to harbor stem cells, i.e., the subventricular zone (SVZ) of the lateral ventricle. Single cell suspensions of the microdissected mouse brain tissue were then used for identification of characteristic events of SVZ neurogenesis in vitro.

To minimize the presence of more mature SVZ phenotypes and to expand the putative stem cell population at the same time, SVZ cultures were passaged twice before experimentation (representing approximately 5 population doublings) in media known to maintain and promote proliferation of neural stem cells. As described above, media supplements included epidermal growth factor (EGF), basic fibroblast growth factor (bFGF), serum, and N2-components. This permitted monolayer proliferation of isolated cells in adhesive conditions without apparent loss of cells due to the formation of floating cellular aggregates or cell death.

For experimental analysis, cells were seeded onto glass coverslips coated with laminin and poly-L-ornithine and evaluated at near confluency. Referring to FIGS. 1A and 1B, at passage 3, proliferating SVZ cultures included two major cell populations. Large protoplasmic astrocytes expressing glial fibrillary acidic protein (GFAP) were found growing on top of underlying small cells with trapezoidal morphologies. The underlying population, representing approximately two-thirds of all cells in this condition, was labeled with the monoclonal antibody A2B5 that recognizes an immature neural ganglioside, and was positive for nestin, an intermediate filament found in neural precursor cells. Interestingly, many of these cells, which were trapezoidal in shape, were also found to co-express some GFAP filaments (FIG. 1A, arrowhead). Neuronal markers (for example, β-III tubulin, Map-2, NeuN) or oligodendroglial markers (for example, 04, CNPase) were not detected among the underlying cell population.

Ultrastructural characterization of trapezoidal cells revealed elongated nuclei embedded in light flowing cytoplasm, with large numbers of mitochondria and free ribosomes, as well as intermediate filaments (FIGS. 1C and 1D).

Whole cell patch-clamp recordings of proliferating trapezoidal cells displayed a predominance of A-type K⁺ currents (IK_{A}) with hyperpolarized resting membrane potentials (Vᵣ, −740±2.6 mV; n=45), a membrane capacitance (Cₘ) of 33.8±4.3 pF, and low input resistance (Rᵢₙ, 166.2±33.0 MΩ) (FIG. 1E). None of the recorded proliferating cells was capable of generating action potentials, but a small percentage (13.3%, 6 out of 45) possessed tetrodotoxin (TTX)-sensitive voltage-gated sodium channels. Together, these findings suggested that particularly the trapezoidal population among cultured SVZ cells had immature glial characteristics.

### Example 3

### Stage I of In Vitro Neurogenesis: Characterization of SVZ Progenitor Cells (“Phase Dark” Cells)

The simultaneous removal of FGF, EGF and serum from proliferating cultures of postnatal day 8 (P8) and adult (>90 days old) SVZ reliably induced the appearance of defined colonies of 10 to 100 phase dark cells 4 days later (FIG. 1F). This phenomenon was not observed during proliferation of SVZ cells, consistent with the finding that EGF arrests cell differentiation (Doetsch F. et al., Neuron 36:1021, 2002). Identically cultured parietal neocortex cells did not yield phase dark clusters, nor did SVZ cells propagated in medium lacking growth factors.

Phase dark cells were GFAP⁻ and A2B5⁻, yet expressed nestin and β-III tubulin. Referring to FIG. 1G, many of these cells also expressed both Dlx-2 and PSA-NCAM. In close proximity to these cells were other clusters of cells that were positive for Dlx-2 but negative for PSA-NCAM.

As seen in FIG. 1H, ultrastructural investigation revealed small cells (approximately 5 μm in diameter) of oval or rounded morphology, with a high nucleus/cytoplasm ratio, and characteristically dense, sometimes invaginated nuclei. Directly underlying the newborn cell clusters were trapezoidal cells, shown in FIG. 1I. This relationship suggests that a close association is necessary for the in vitro formation of phase dark cells.

Electrophysiological recording by whole cell patch clamping (FIG. 1J) revealed that upon formation, phase dark cells (n=20) displayed membrane properties similar to those of SVZ-born neural progenitors characterized in vivo (D. D. Wang, D. D. Krueger, A. Bordey, J Neurophysiol 90, 2291, 2003). Compared to proliferating SVZ cells (FIG. 1E), phase dark cells were significantly more depolarized (i.e., −18±1.6 mV) with very low Cₘ (6.8±0.51 pF) and very high Rᵢₙ (4.6±0.72 GΩ). Significant sodium channel contribution was not observed in the phase dark cells.

The membrane properties displayed by the phase dark cells are similar to those of SVZ-born neural precursors characterized in vivo (Wang D. et al., J. Neurophysiol. 90:2291, 2003). SVZ neurogenesis proceeds as a characteristic series of events, where multipotent glial cells (referred to as type-B cells) are capable of dividing to form colonies of neuroblasts (type-A cells) through a transit-amplifying cell population (type-C cells) (Doetsch F., Nat. Neurosci. 6:1127, 2003). Newborn neuroblasts migrate from the SVZ through the rostral migratory stream and mature to GABAergic granule cells and periglomerular cells, which integrate as inhibitory interneurons into the olfactory bulb of rodents. The combined antigenic, ultrastructural and functional profiles of the phase dark cells described herein correspond to those that are distinctive for stem cell-generated neuroblasts and transit-amplifying cells found in the rodent SVZ (Doetsch, F. et al., J. Neurosci. 17:5046, 1997; Doetsch, F. et al., Neuron 36:1021, 2002).

### Example 4

### Stage II of In Vitro Neurogenesis: Morphological and Functional Characterization of Maturing Newborn Neurons

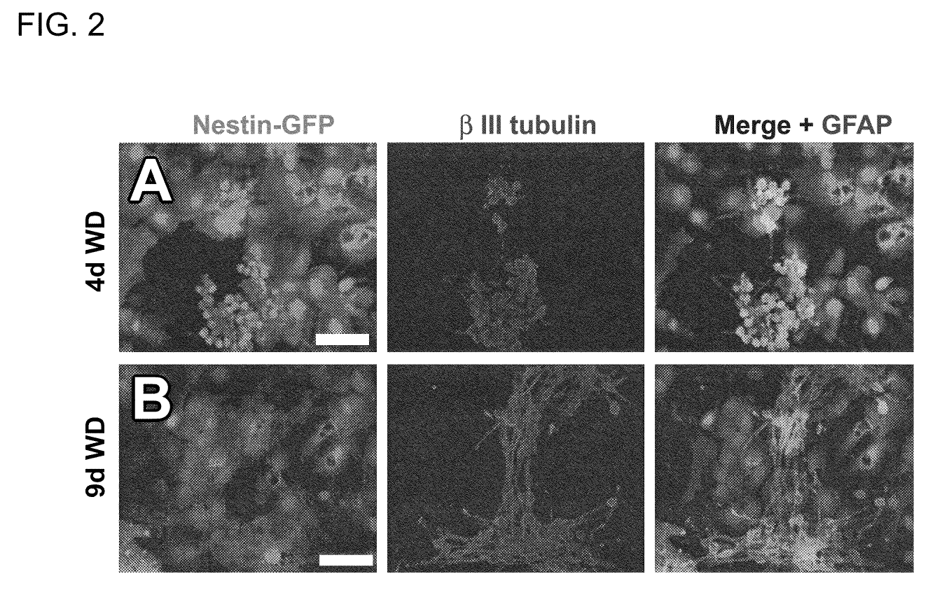

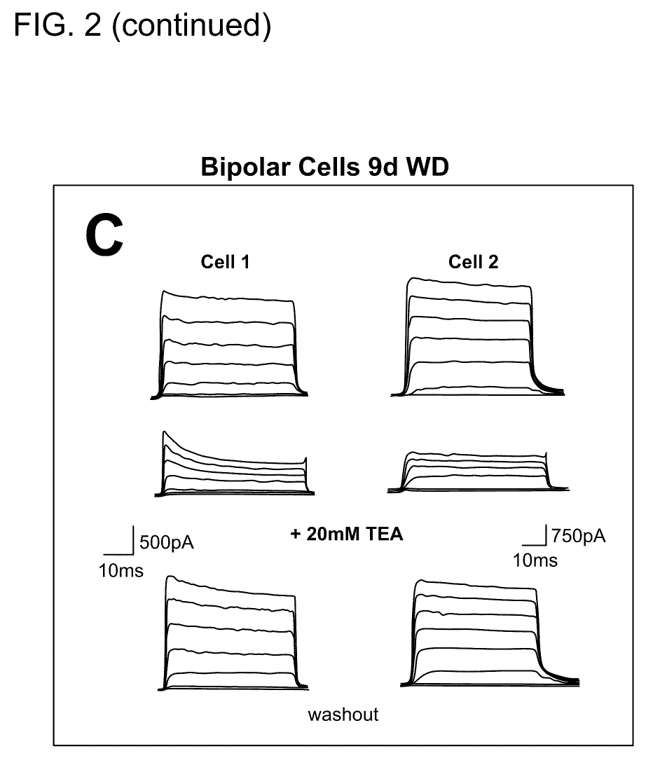

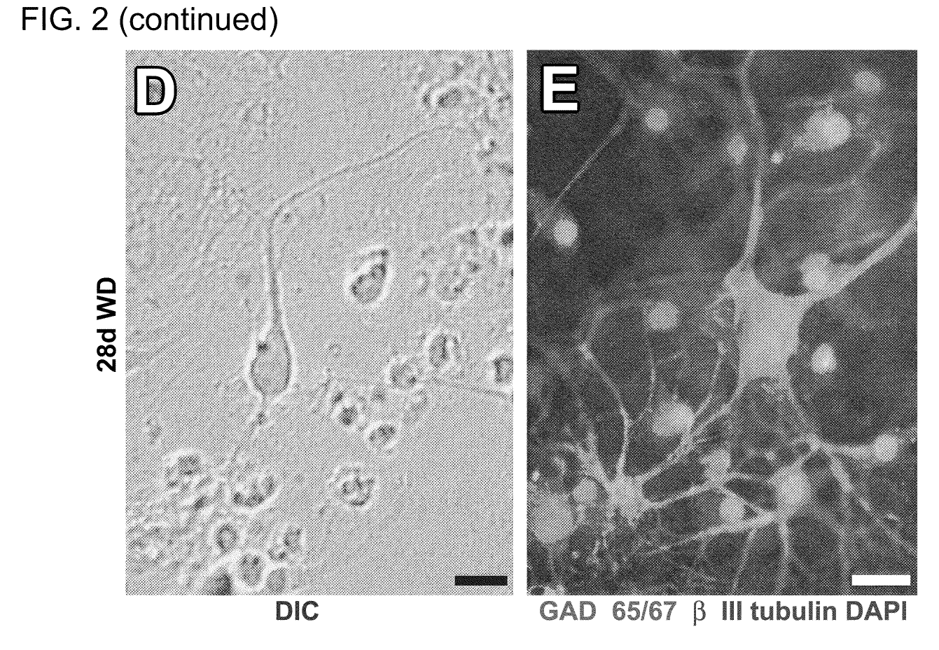

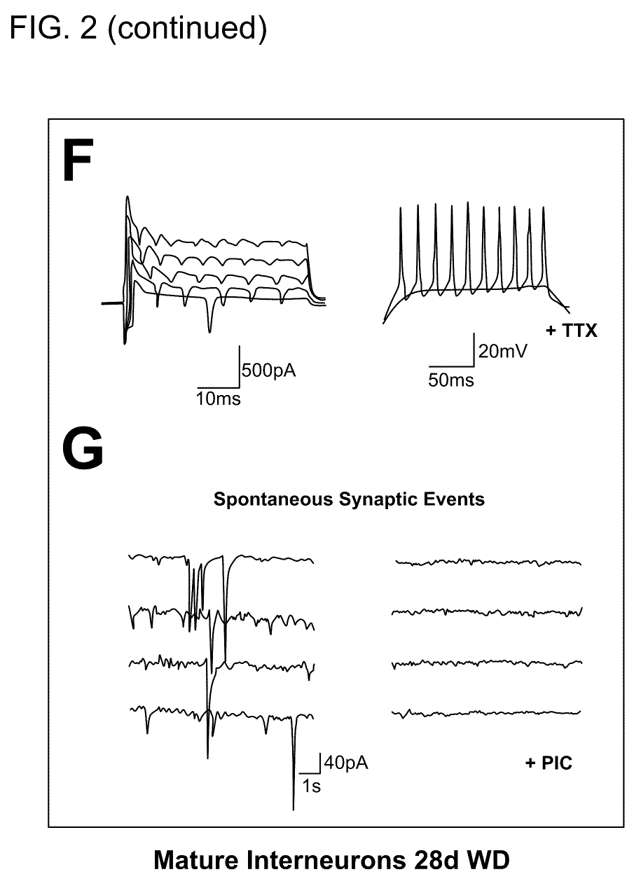

In vitro fate analysis was used to determine whether the appearance of phase dark cells in culture represented a characteristic event of SVZ-specific neurogenesis. We induced terminal differentiation of SVZ cultures using retinoic acid (see Methods) and longitudinally tracked isolated colonies of phase dark cells for morphological, immunological, and biophysical development. Referring to FIG. 2A, phase dark cells initially co-express nestin and β-III tubulin (FIG. 2A, merged image), however these cells soon downregulate nestin and begin to extend bipolar processes (FIG. 2B).

In comparison to membrane properties of phase dark cells, these bipolar cells (n=20, 9±2 days after growth factor withdrawal) become more polarized (Vₘ, −47±5.0 mV), Cₘ, increases (11.8±0.54 pF), and Rᵢₙ, decreases (1.1±0.322 GΩ). Similar to previous in vitro studies (Stewart, R. R. et al., J. Neurophysiol. 81:95, 1999) and similar to recordings from migrating neuroblasts in vivo (Belluzzi, O. et al., J. Neurosci. 23:10411, 2003), the bipolar cells display delayed rectifying K⁺ currents (1K_{dr}) which are sensitive to application of 20 mM tetraethylammonium (TEA, FIG. 2C). Interestingly, in some bipolar cells, application of TEA exposed underlying IK_{A}, the typical potassium current of proliferating (immature/glial) SVZ cells (see FIG. 1E; compare cell 1 and 2 in FIG. 2C).

No sodium-mediated currents were observed at this stage. With time in culture, neuritic arborisation became increasingly complex, and markers of more mature neurons (i.e., Map-2, NeuN) were expressed. Four weeks after growth factor withdrawal, bipolar cells had differentiated into characteristic mature neuronal phenotypes (FIGS. 2D and 2E). These neurons almost exclusively expressed antigens for glutamic acid decarboxylase (GAD), the key enzyme of intracellular synthesis of gamma-aminobutyric acid (GABA). (See FIG. 2E.)

Whole-cell patch clamp recordings (n=14) at 28±4 days after growth factor withdrawal demonstrated in comparison to bipolar cells lower Vₘ (−48.1±7.2 mV), decreased Rᵢₙ (583.6±183.8 MΩ), and increased Cₘ (21.4±2.5 pF). Referring to FIG. 2F, TTX-sensitive repetitive action potentials could be elicited at this developmental stage, suggesting terminal differentiation of SVZ culture-derived neurons. Mature neurons (6/6 recorded cells) displayed spontaneous synaptic activity that surprisingly consisted almost exclusively of inhibitory events, as demonstrated by application of picrotoxin, an inhibitor of GABA-mediated synaptic transmission (FIG. 2G). The combination of morphological and functional findings implies that phase dark cells develop primarily into GABAergic interneuron phenotypes in vitro over a protracted period of time, thus recapitulating SVZ neurogenesis in the absence of developmental cues from the brain.

### Example 5

### Characterization of a Mitotically Active Multipotent SVZ Precursor Cell that Emerges Transiently in Stage I of In Vitro Neurogenesis

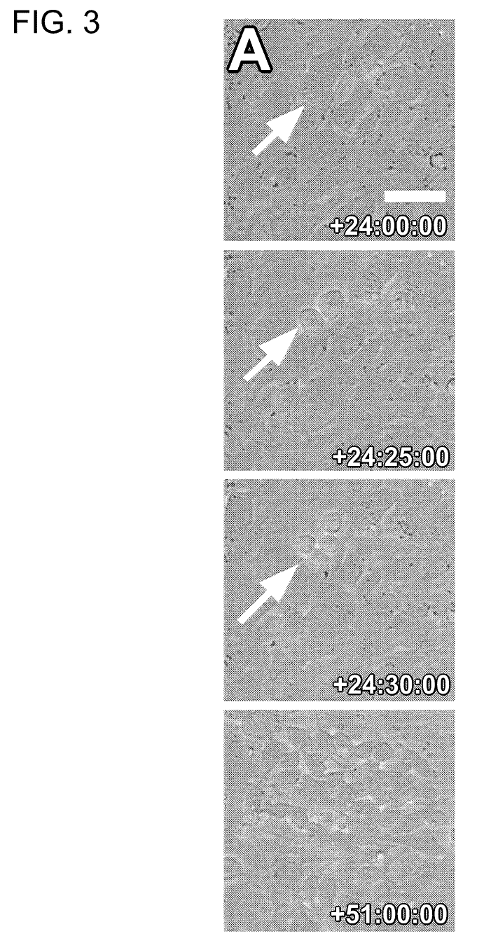

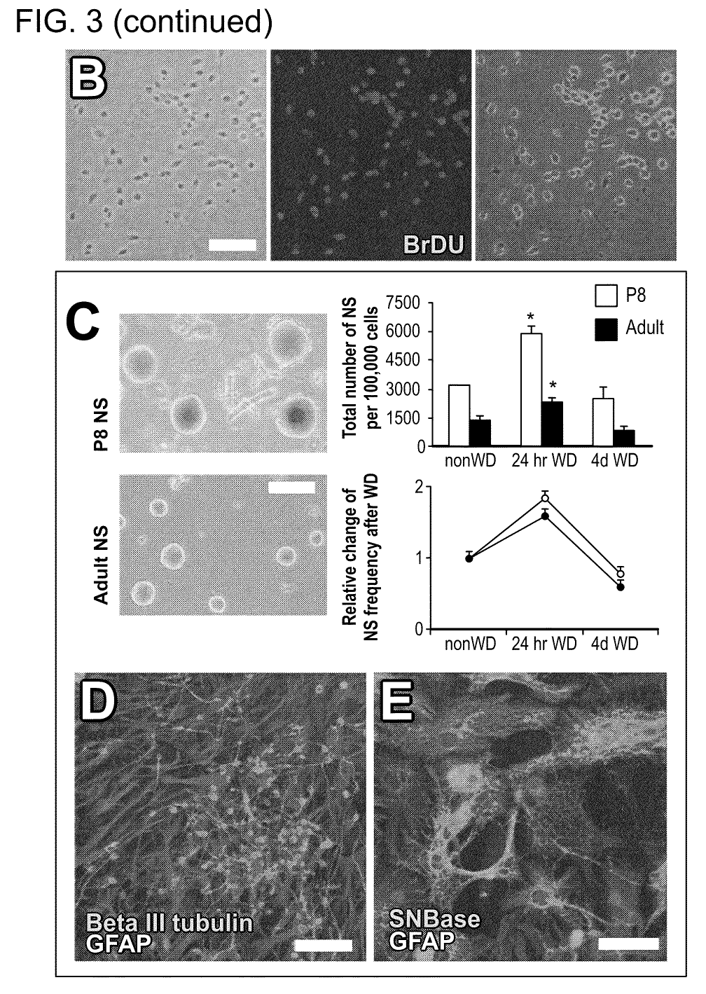

Consistent with findings from in vivo studies, the data described herein indicate that SVZ neurogenesis proceeds in two stages, and that the transition of glial-like cells to neuroblasts occurs within the first 96 hours. Real-time microscopy was employed to further capture and characterize the immediate dynamics of developing SVZ cells. Referring to FIG. 3A, after withdrawal of growth factors and serum from proliferating SVZ cultures, cells maintain a flat, amorphous-glial appearance for one day. After this “silent period,” a sudden and widespread series of rapid cell divisions is observed. The cell divisions are accompanied by dramatic morphological transformations, ultimately leading to the initial appearance of phase dark cells as early as 16-24 hours later. The timing of this rather remarkable event is consistent with in vivo observations reporting that 50% of putative SVZ stem cells are mitotically active two days after depleting the constitutively proliferating cells of this brain region.

Time-lapse observations also confirmed the approximated 12.7-hour cell cycle time reported for the rapidly dividing SVZ population in vivo. After their initial appearance, phase dark cells become compact and form clusters, which are widespread by 4 days following induction of differentiation. During this time, cell divisions continue, as revealed by birth dating experiments using 5-bromo-2-deoxyuridine (BrdU). Referring to FIG. 3B, more than 90% of phase dark cells take up BrdU when labeled 48-72 hours after growth factor withdrawal. Application of BrdU between 72 and 96 hours labels only few phase dark cells, suggesting that the period of mitotic activity is transient.

These findings clearly reveal that developing SVZ precursor cells accomplish a series of profound morphogenic changes in a surprisingly short period of time. It was of interest to determine how these observations correlate with the more conventional neurosphere assay (Reynolds and Weiss, Science 255:1707, 1992; Kukekov et al., Exp. Neurol. 156:333, 1999). As each neurosphere (NS) represents one clonally derived cellular aggregate that can give rise to both neurons and glia, and since some cells of these dissociated NS form secondary NS, this assay represents a “post hoc” analysis of “stemness” (clonogenicity, multipotency, self-renewal) in a given cell population.

Applied to our paradigm (see Methods), the NS assay exposed a transient increase in numbers of NS at 24 hours after withdrawal of growth factors, as shown in FIG. 3C. This change was significant and profound, as NS numbers almost doubled (1.86 and 1.6 times in P8 and adult, respectively) compared to proliferating SVZ cultures, and dropped to below initial levels at 4 days after withdrawal. Interestingly, although adult SVZ cultures generate fewer total numbers of NS than P8 dissociates, the relative change of NS frequencies was comparable. We also noted a difference between P8 and adult SVZ-derived NS diameters. However, for both age groups and at each investigated time point, dissociated primary NS yielded clonally derived secondary NS, suggesting a profound potential for self-renewal. Referring to FIGS. 3D and 3E, plated primary and secondary NS were confirmed to give rise to neurons and glia (evidenced by positive immunoreactivity of cells for β-III tubulin and CNPase, respectively), validating the multipotency of clonogenic isolates.

### Example 6

### Characterization of a Rapidly Dividing Intermediate Cell Type (“Teardrop Cell”) Intermediate Between Stem-Like Glial Cells (Immature Precursor Cells) and Phase Dark SVZ Progenitor Cells

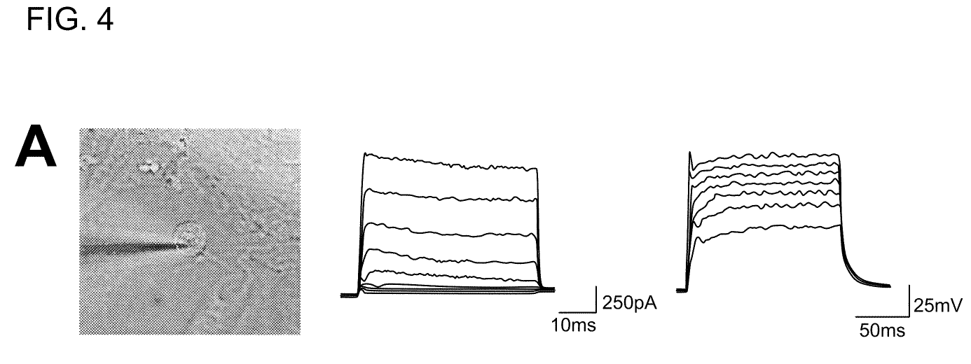

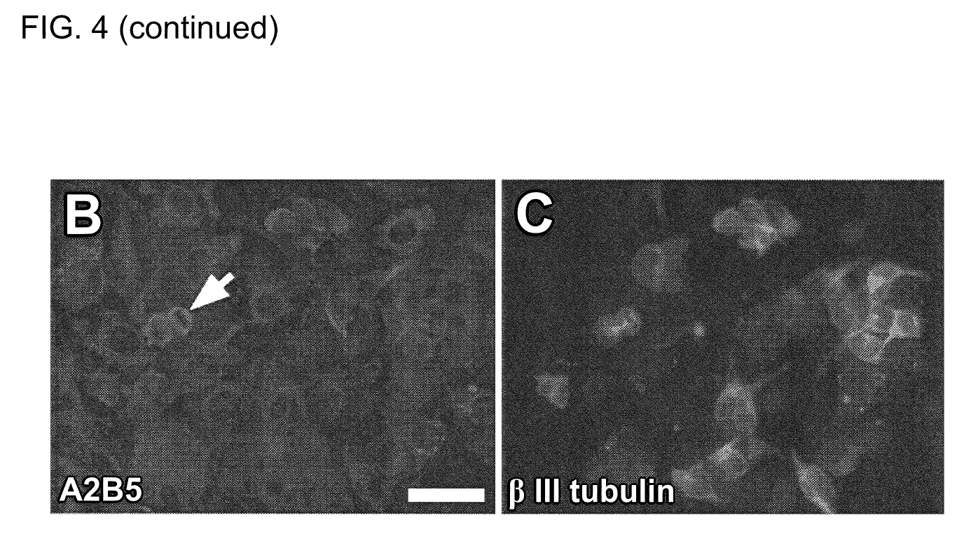

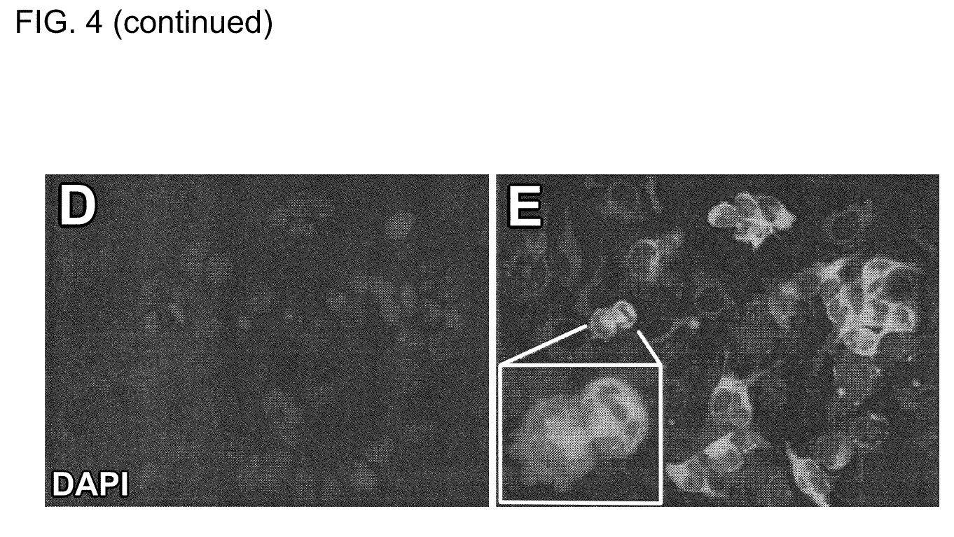

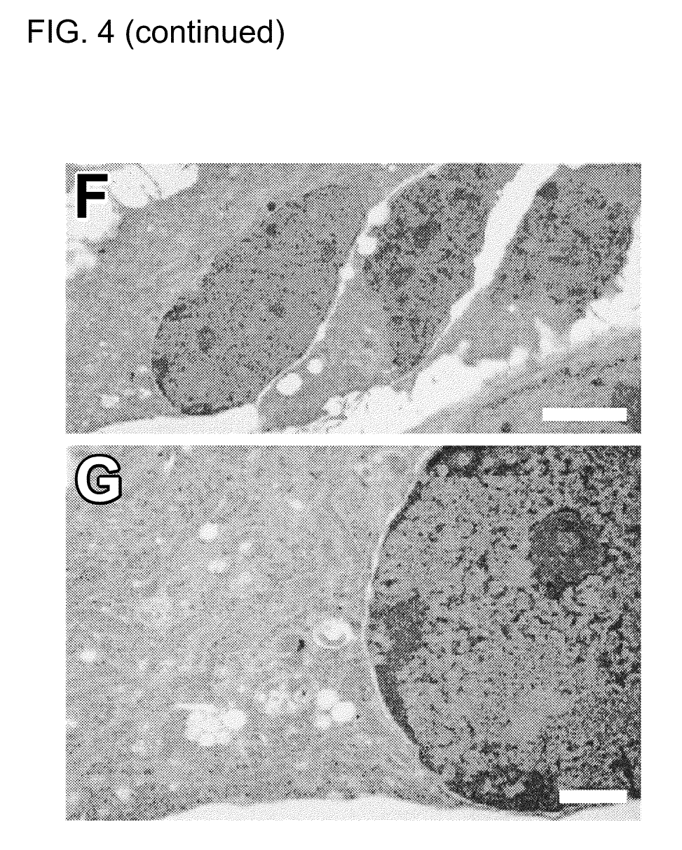

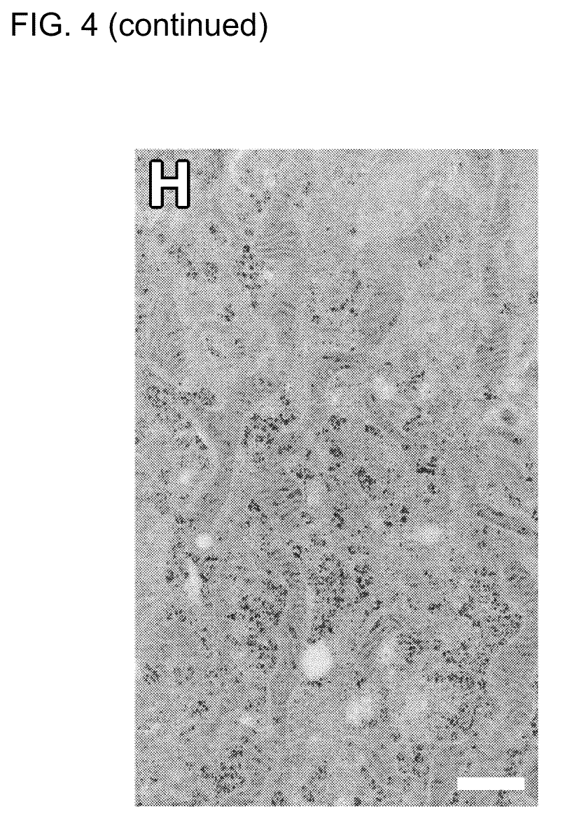

Taken together, the findings attribute “sternness” to the rapidly dividing cell population as described above. To further investigate and distinguish this novel cellular phenotype, we isolated and examined passage 3 SVZ cells one day following growth factor withdrawal. Patch-clamp analysis of these cells was performed at the time of cell division (FIG. 4A) or shortly thereafter (n=8). The results revealed passive membrane characteristics intermediate between those of proliferating SVZ and the phase dark cells (Vₘ=−60±8.4 mV; Cₘ=12.5±1.3 pF; Rᵢₙ=661±162.6 MΩ). Interestingly, this transient population exhibits low potassium conductances and a predominance of IK_{dr} (characteristic for phase dark and bipolar cells), as opposed to the typical IK_{A} exhibited by proliferating cells (compare FIG. 4A with FIGS. 1J and 2C). No sodium channel-mediated currents were observed.

Immunophenotyping of these cells reveals strongly A2B5-expressing mitotic cells (FIG. 4B, arrow) located in close proximity to small clusters of distinctive β-III tubulin positive cells (shown in FIG. 4C). This teardrop-shaped cell population inconsistently expresses A2B5, and displays condensed, bright DAPI (4, 6-dianidino-2-phenylindole)-labeled nuclei (FIG. 4D). These cells rarely express Dlx-2, and are consistently negative for PSA-NCAM and GFAP. Interestingly, the “teardrop cell” described herein, transient in our culture model, has been acknowledged to exist in our previous in vivo studies of the SVZ of adult rodents (Gates, M. A. et al., J. Comp. Neurol. 361:249, 1995), but the implications of this finding were not appreciated at that time. The findings described herein indicate that these cells are multipotent and intermediate between stem-like glial cells and phase dark progenitor cells.

The expression of β-III tubulin, particularly in association with mitotic spindles (FIG. 4E, inset) is unique. Without wishing to be bound by theory, it is believed that this finding may offer a clue as to the transient nature of this cell type/developmental window, since the promoter region of the β-III tubulin gene contains responsive elements to AP2 and MATH-2. These elements are implicated in glial/neuronal fate choices and cell cycle regulation.

Referring to FIGS. 4F and 4G, electron microscopy of teardrop cells (FIG. 4F) reveals prominent vacuoles (FIG. 4G). Large numbers of mitochondria and free ribosomes are seen in the cytoplasm, (FIG. 4H), but intermediate filaments are lacking.

### Example 7

### Neuropoiesis as Recapitulated In Vitro Models

We provide compelling evidence based on the disclosed studies that subventricular neurogenesis can be isolated and recreated in vitro. As much as it is surprising that characteristic neurogenic events can be accurately recapitulated away from the developmental cues of the brain, the use of simplistic culture models has been shown to be instrumental in uncovering functional aspects of neural stem cell biology (see, for example, Shen Q. et al., Science 304:1253, 2004).

The pioneering work of Dexter et al. (J. Cell Physiol. 91:335, 1977) showed how cells from bone marrow can establish themselves in adhesive culture conditions that mimic specific environmental cues for the proliferation and differentiation of haematopoietic cells. Our model and results presented herein further support similarities between hematopoiesis and neuropoiesis, which we have previously noted (Scheffler B et al. Trends Neurosci. 22:348, 1999).

As the SVZ represents one major neurogenic niche of the adult brain, which we collectively refer to as “brain marrow,” and as neuropoiesis refers to brain marrow-specific events, it is of interest to directly observe phase dark cells emerging through a series of mitotic events rather than representing the result of one single cell division. This observation is in keeping with the view that stem cell-driven cellular replenishment in the postnatal organism occurs mainly through transit amplifying cell populations (Potten et al., Development 110:1001, 1990). At the same time, our observations highlight dissimilarities to embryonic development, in which radial glial cells generate cortical neuroblasts directly, via one asymmetric cell division (Miyata T et al., Neuron 31:727, 2001).

In addition to visualizing and characterizing the metamorphosis of stem-like glial cells, we made the surprising finding of the rapidity of transition from glial to neuronal phenotype. Within 24 hours, proliferating glial cells (characterized as GFAP^{low+}/A2B5⁺/Nestin⁺/Dlx-2⁻/β-III tubulin⁻) give rise to rapidly dividing intermediate cells (characterized as GFAP⁻/A2B5^{+/-}/Nestin⁺/Dlx-2^{+/-}/β-III tubulin⁺). Intermediate cells in turn become phase dark cells, also termed neuroblasts (characterized as GFAP⁻/A2B5⁻/Nestin⁺/β-III tubulin⁺/Dlx-2⁺/PSA-NCAM⁺) 3 days later. Discovery of the rapid morphogenic cadence during early stages of neuropoiesis demonstrated herein may provide an explanation for the apparent elusiveness of stem cells in the adult brain.

### Example 8

### Materials and Methods

The following materials and methods were used in Examples 9-13 below, which describe several variations of the system and methods of the invention.

Animals. The experiments described below were performed using postnatal day 8 and adult (>60 days of age) mouse brain tissue. Wild-type C 57/B6 and Nestin-GFP transgenic C57B1/6×Balb/cBy hybrid mice were used.

Derivation of cells. Day 1: mice were decapitated and brain tissue from the SVZ was microdissected rapidly and transferred into ice-cold DMEM/F-12 media (DF; Invitrogen 11320-033) supplied with 2% antibiotic-antimycotic (abx; Invitrogen 15240-062) solution. Tissue of the rostral and posterior regions of the subventricular zone (SVZ) was microdissected, trypsinized, and cultured in plastic dishes overnight in DMEM/F-12 media containing N supplements, 5% serum, EGF, and bFGF (20 ng/ml each).

More specifically, non-adherent cells were harvested and propagated under several different conditions, i.e., under conditions that permit formation of monoclonal neurospheres (as described in Kukekov et al., 1997), in high-density suspension cultures which result in formation of polyclonal neurospheres, and as adherent monolayers.

The brain tissue was shown to remain viable for up to 48 hours under each of these conditions. Under aseptic conditions, the tissue was minced using scalpels to small (about 1 mm³) chunks, washed twice in PBS (Dulbecco's Phosphate-Buffered Saline; Invitrogen 14190-144) containing 1% abx, and transferred to 0.25% balanced trypsin solution for 20 min at 37° C. Fetal calf serum (FCS; Hyclone SH30071.03) was added to a final concentration of 1%, and the tissue was triturated through fire-polished glass pipettes of decreasing widths until a single cell suspension was obtained. The cellular suspension was centrifuged, and cells were then resuspended in N5 media (DF, 5% FCS, modified N-components [N-components include 100 μg/ml human apo-transferrin, 5 μg/ml human insulin, 20 nM progesterone, 30 nM sodium selenite, 100 μM putrescine], and 1% abx) supplemented with 40 ng/ml each of EGF and bFGF and 37 μg/ml pituitary extract. Cells were placed into untreated plastic cell culture dishes or flasks at a density of at least 50,000 cells/cm² and cultured overnight in a humidified 37° C. incubator with a 5% saturated CO₂ environment.

Day 2: The following day, non-adherent cells were collected from the tissue culture dishes/flasks, and a single cell suspension was prepared by trituration with glass pipettes as described above, either with or without trypsin digestion. The cells were then added at a density of at least 50,000 viable cells/cm² into wells of 6 well plates (Costar (#3516) in N5 media containing 40 ng/ml each of EGF and bFGF. The cells were then cultured overnight in a humidified 37° C. incubator with a 5% saturated CO₂ environment.

Post day 2: The expansion of multipotent cells from postnatal brain started at day 2 in culture. EGF and FGF were added (20 ng/ml each) on a every other day until the cells became confluent. Cells generally reached a high confluent level after 1-4 days under these conditions, and they were then serial-passaged at 1:2 dilutions. If cells in the culture were not confluent by the 4^{th} day, the medium was replaced and the culturing was continued. Cells were passaged by removing the adherent cells using trypsin, and then seeding these cells at densities between 75,000 and 85,000 viable cells/cm² in new culture dishes/flasks. After passaging and at medium changes, EGF and FGF were supplied at concentrations of 40 ng/ml, and at concentrations of 20 ng/ml bidaily thereafter.

### Example 9

### Cryopreservation and Expansion of Proliferating Neural Precursor Cells

At any given stage during the proliferation phase, cells can be frozen and stored in liquid nitrogen for extended periods of time. The cells can then be thawed and can undergo the same treatment schedule, yielding the same expansion rates of non-frozen specimens without losing their stem cell attributes. For example, cells frozen at passage 1 were later able to be expanded through serial passaging to passage 20. These cells were identical in behavior to cells derived from wild type C57/B6 mice serial passaged similarly without freezing.

### Example 10

### Induction of Phase Dark Cells In Vitro Following Growth Factor Removal

The following observations were made using methods described in Example 8 above, which was found to be suitable for isolating and studying inducible, differentiable, and self-renewing neural precursor cells from postnatal brain. Under culture conditions containing serum, N-components, and the growth factors bFGF and EGF, described above, mouse SVZ-derived cells were maintained as neurospheres or in adhesive monolayers. Upon removal of mitogenic factors from neurospheres grown in suspension cultures, clusters of small phase-dark cells that co-expressed nestin and β-III tubulin emerged.

A comparison was made of the presence and relative quantity of phase dark cell clusters appearing after withdrawal of growth factors using rostral or posterior brain tissue derived from P8 and adult (greater than 60 days of age) mice. The results showed that using monoclonal neurosphere conditions, no phase dark cells could be obtained from the adult brain or from rostral P8 brain, and a few such clusters could be obtained from posterior P8 brain tissue. Using polyclonal neurospheres, small numbers of phase dark clusters, comparable to the numbers obtained from monoclonal neurospheres (P8, posterior), were obtained form all four tissue sources. By contrast, adhesive culture conditions were conducive to production of large numbers of phase dark cells from adult rostral tissue, and even greater numbers of these cells from both rostral and posterior SVZ brain tissue from P8 animals. Most notably, adherent monolayers remained able to generate small phase dark cells after one and five passages in culture.

Studies using BrdU uptake (10 μM×24 hr) demonstrated that the small phase-dark cells appeared in the cultures 48-72 hours after withdrawal of growth factors. Serial passaging experiments and BrdU analysis revealed that the small phase dark cells were consistently “born” 2-3 days following growth factor withdrawal from proliferating monolayer cultures regardless of prior passaging.

Newly-born phase-dark cells expressed immature neuronal markers. For example, clusters of phase-dark cells appearing 3 days following growth factor withdrawal from passage 5 cultures were seen to express PSA-NCAM, nestin and β-III tubulin. No GFAP expression was observed.

### Example 11

### Maturation of Phase Dark Cells in Adherent Cultures

Upon maturation, the phase dark cells lost nestin expression and extended processes, adopting a more mature neuronal morphology. More specifically, upon prolonged withdrawal of serum and growth factors, round to oval phase-dark cells matured into bipolar, highly migratory active phase bright morphotypes, reminiscent of immature neuroblasts. At 1 day after appearance, they co-expressed nestin and β-III tubulin. Observed seven days later, the cells had migrated away from their “birth cluster,” and at this stage, no longer expressed nestin but continued to express β-III tubulin.

To determine whether neuronal precursors were the only cell type generated upon growth factor withdrawal, we characterized the monolayer under proliferative conditions. More particularly, under phase examination, proliferating monolayers of cells revealed morphological similarities to astrocytes, and ubiquitously co-expressed nestin and GFAP. A minor, but distinct subpopulation of proliferating cells with peculiar morphologies was also present that expressed β-III tubulin. Some GFAP astrocytes also co-expressed vimentin. Markers of mature oligodendrocytes and neurons (e.g., CNPase and MAP2, respectively) were not present under these conditions.

A profile of the underlying cell layer in adherent monolayers after growth factor withdrawal was also obtained. It was found that discontinuation of EGF, bFGF and serum supply induced the appearance of CNPase-expressing oligodendrocytes in areas surrounding the phase-dark cell clusters. Similarly, an increase of vimentin-expressing cells was observed. Additionally, many of the underlying cells expressed GFAP and nestin, and a few expressed β-III tubulin. Results of the immunotyping demonstrated that a broad variety of different cell subpopulations was present among adherent cells after growth factor withdrawal.

### Example 12

### Maintenance and Expansion of Cells in Adherent Culture Conditions

The use of untreated plastic culture dishes, flasks, or 6 well plates enhanced the proliferation rate and developmental multipotency of the cultured cells, whereas the use of other surfaces (e.g., 0.1% gelatin or laminin [1 ug/ml]/poly-L-ornithine [15 ug/ml]) reduced the proliferation rate and developmental potential of the cultured cells. High cell densities favored the maintenance and expansion of multipotent cells under adherent culture conditions. For example, under the specific conditions described above, after the initial plating at 50,000 viable cells/cm² on day 2, further serial passaging of the cultures appeared to require starting with between 75,000 and 85,000 viable cells/cm². The use of lower cell densities had negative effects on the maintenance of multipotentiality and cell cycle time.

Media: In the experiments described above using DF medium, the inclusion of the N-components facilitated the culture process. Further, as described above, cells could be propagated in media supplemented with 5% FCS for up to 15 passages without losing their multipotentiality. Cells expanded in 5% FCS showed a significant delay in differentiating into neurons and glial phenotypes in response to inductive events. For example, in 5% serum-containing media, in initial passages, differentiation occurred in only 2-3 days. In comparison, in passage 15, differentiation was not noted until up to 14 days of culture. Interestingly, after the second day of culture, serum did not appear to be required for the further expansion of the multipotent brain cells.

In some experiments, multipotent cells were successfully expanded in adhesive conditions when FCS was removed following day 1 of culture. Cells cultured in serum-free media (N-media [DF, modified N-components, and 1% abx] supplemented with EGF and bFGF) showed a rapid (reliably 2-3 days even at higher passages) response to inductive stimuli of spontaneous differentiation; however, they required a different procedure for the induction process (see Example 13 below).

EGF also appeared to be required for the expansion of multipotent cells in adherent culture conditions. This growth factor was supplied to the proliferation media initially at 40 ng/ml, and then added every other day at a concentration of 20 ng/ml. Cells cultured in EGF alone proliferated continuously with doubling times of 5-6 days, and retained their multipotentiality—exhibiting a large number of oligodendrocytes among the newborn neuronal and glial progeny upon differentiation. In comparison, bFGF was optional for the expansion of multipotent cells in adherent culture conditions. Cells in cultures supplemented with bFGF and EGF (both at 40 ng/ml initially and then 20 ng/ml/(every other day), showed a high proliferation rate (doubling times average 36 hours, constant over at least 20 passages) and retained their multipotentiality—differentiating into a large number of neurons among the newborn neuronal and glial progeny. Interestingly, cells ceased to proliferate within 2-3 passages when the media was supplemented with bFGF (10-40 ng/ml) without EGF. Under these conditions, very few newborn neurons and glia were observed upon induction of differentiation. The influence of PDGF (platelet-derived growth factor), and LIF (leukemia inhibitory factor) was also examined in this system, but were not found to benefit either the maintenance or the quality of expanded cell population.

### Example 13

### Induction of Differentiation in Adherent Culture Conditions

Cells proliferating in N5 media supplemented with FGF and EGF were induced to differentiate into neurons, astrocytes, and oligodendrocytes as described above. More specifically, differentiation was induced by the withdrawal of serum, FGF, and/or EGF. This was achieved by removing the N5 media supplemented FGF and EGF, washing the cells two times with PBS, and then adding N-media to the cells without the serum and/or growth factors. All three factors have to be withdrawn simultaneously. Sequential withdrawal of serum followed by growth factor withdrawal did not result in differentiation, nor did growth factor withdrawal followed by subsequent serum withdrawal cause differentiation.

Most of the inductions were performed on poly-L-ornithine (15 μg/ml/laminin (1 μg/ml)-coated surfaces (glass coverslips). Such surfaces have shown to provide an ideal substrate for cell attachment, neural differentiation, and maturation. Use of untreated substrates (for example, glass or plastic alone) resulted in lack of cellular attachment and/or significantly decreased quantities of inducible neuroblasts. Successful induction of differentiation can be assessed by light microscopy. For example, the appearance of small, phase-dark cell clusters (neuroblasts) on top of large, flat underlying cells indicates that differentiation has been induced. While the presence of neuroblasts indicates that differentiation has been induced, newborn oligodendrocytes and astrocytes are found among the underlying cell layer. The time between the inductive event and the appearance of neuroblasts increases corresponding to (passage) time in culture. Neuroblasts generally appear 1-2 days after induction at passage 1, 3-4 days after induction at passage 5, and 14 days after induction for passage 13.

Cells first passaged extensively in serum-containing medium that are then switched to serum-free medium give rise to small phase-dark cells more rapidly (5 days) than their serum-cultured counterparts (14 days). Differentiation of cells cultured in serum-free conditions (N5 media supplemented with EGF and FGF) is induced by first culturing the cells for at least 1 day in N5 supplemented with EGF and FGF and thereafter removing the serum, EGF, and FGF. After induction in this manner, neuroblasts appear in 2-3 days at both the first and fifth passages.

### Other Embodiments

It is to be understood that while the invention has been described in conjunction with the detailed description thereof, the foregoing description is intended to illustrate and not limit the scope of the invention, which is defined by the scope of the appended claims Others aspects, advantages, and modifications are within the scope of the following claims.

1. A method for culturing neural precursor cells comprising the steps of:
(a) isolating tissue comprising neural precursor cells from an animal subject;
(b) dissociating the tissue to single cells; and
(c) attaching the single cells to a substrate in medium comprising pituitary extract and mitogenic factors EGF and bFGF, for a duration of time sufficient to produce a culture comprising at least one cell type that is proliferating and/or differentiated.

2. The method of claim 1, further comprising the step (d) of culturing the cells in medium lacking mitogenic factors.

3. The method of claim 1, wherein the medium comprises N2 components.

4. The method of claim 1, wherein the cell type is an immature precursor cell that expresses phenotypic markers of both neural stem cells and glial cells, but not markers of neuronal cell lineage.

5. The method of claim 4, wherein the immature precursor cell is passaged in culture.

6. The method of claim 2, wherein the cell type is a rapidly dividing intermediate cell that is produced in culture about 1 day after withdrawal of mitogenic factors, and expresses both phenotypic markers of neural stem cells and phenotypic markers of neuronal lineage, but not the glial cell marker GFAP.

7. The method of claim 6, wherein the cell expresses the neural stem cell marker nestin, the immature glial cell marker A2B5, and the early neuronal cell marker β-III tubulin.

8. The method of claim 6, wherein the intermediate neural progenitor cell expresses nestin and markers of early neuronal lineage comprising β-III tubulin and Dlx-2, but not markers of later neuronal lineage including MAP2, NeuN and GAD.

9. The method of claim 2, wherein the cell type is a SVZ progenitor cell that is produced in culture about 2-4 days after withdrawal of mitogenic factors, and expresses both phenotypic markers of neural stem cells and phenotypic markers of neuronal lineage, but not the glial cell marker GFAP.

10. The method of claim 9, wherein the SVZ progenitor cell expresses nestin and markers of early neuronal lineage comprising β-III tubulin and Dlx-2, and at least one marker of late neuronal lineage selected from the group consisting of MAP2, NeuN, and GAD.

11. The method of claim 2, wherein the medium in step (d) further comprises retinoic acid, added to the culture medium following withdrawal of mitogenic factors, to induce differentiation of neurons.

12. The method of claim 11, wherein the cell type is a differentiated neuron capable of generating an action potential.

13. The method of claim 11, wherein the cell type is a bipolar cell.

14. The method of claim 11, wherein the cell type is a GABAergic neuron that expresses glutamic acid decarboxylase (GAD).

15. The method of claim 1, wherein the cell type is a glial cell.

16. The method of claim 15, wherein the glial cell is an astrocyte or an oligodendrocyte.

17. The method of claim 1, wherein the culture comprises about 40,000 to 80,000 cells/cm² of surface area.

18-27. (canceled)
